(19) United States
(12) Patent Application Publication
(10) Pub. No.: US 2025/0288413 A1
(43) Pub. Date: Sep. 18, 2025

(54) PROSTHETIC HEART VALVE DEVICES

(71) Applicant: MEDTRONIC, INC., Minneapolis, MN (US)

(72) Inventors: Matthew E. GENOVESE, Windsor, CA (US); Mike KRIVORUCHKO, Forestville, CA (US); Gabrielle Marie COLVERT, Huntington Beach, CA (US); Justin G. GOSHGARIAN, Santa Rosa, CA (US); David A. GROSSMAN, Santa Rosa, CA (US); Suryansh KUMAR, Rohnert Park, CA (US); Jordan HARM, Rohnert Park, CA (US); Thuy Linh L. DINH, Westminster, CA (US); Victoria T. TIEN, Apple Valley, MN (US)

(21) Appl. No.: 18/862,999

(22) PCT Filed: May 8, 2023
(86) PCT No.: PCT/IB2023/054765

§ 371 Date: Nov. 5, 2024

Related U.S. Application Data
(60) Provisional application No. 63/342,598, filed on May 16, 2022.
(60) Provisional application No. 63/483,904, filed on Feb. 8, 2023.

(51) Int. Cl. A61F 2/24

(52) U.S. Cl. CPC A61F 2/2418; A61F 2220/0091; A61F 2230/0063; A61F 2230/0067

(57) ABSTRACT

Transcatheter heart valve prostheses include a valve support, a prosthetic valve mounted within the valve support, and an anchoring frame at least partially surrounding the valve support. The anchoring frame and the valve support are attached to each other at respective inflow ends thereof. The anchoring frame includes a fixation portion configured to securely fix the anchoring frame, to tissue at a native heart valve, an integration region configured to integrate the anchoring frame with the valve support, and a lateral portion extending between the fixation portion and the integration region. A brim is coupled to and extends radially outwardly from the anchoring frame at a transition between the lateral portion and the fixation portion.

### FIELD

The present technology is generally related to prosthetic heart valve devices, and in particular is directed to prosthetic heart valve devices for percutaneous repair and/or replacement of native mitral valves and/or native tricuspid valves.

### BACKGROUND

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Transcatheter heart valve prostheses have been developed for repair and replacement of diseased and/or damaged heart valves. Such transcatheter heart valve prostheses can be percutaneously delivered and deployed at the site of the diseased heart valve through catheter-based delivery systems. Such transcatheter heart valve prostheses can be delivered while in a radially compressed configuration so that the valve prosthesis can be advanced through the patient's vasculature. Once positioned at the treatment site, the transcatheter valve prosthesis can be expanded to engage tissue at the diseased heart valve region to, for instance, hold the transcatheter heart valve prosthesis in position.

While these transcatheter heart valve prostheses offer minimally invasive methods for heart valve repair and/or replacement, challenges remain. In particular, when implanted at a native mitral valve prosthesis, obstruction of the left ventricular outflow tract (LVOT) is a concern. Obstruction of the LVOT may result in patient eligibility issues and or risk to the patient for obstruction or restricted blood flow into the aortic valve. Further, native tricuspid valves have a relatively short distance between the native tricuspid valve and the wall of the right ventricle. Native mitral valves and native tricuspid valves may also be referred to generally at atrioventricular valves, as they regulate flow between an atrium of the heart and a ventricle of the heart. For at least the reasons noted above, it is desirable to minimize the distance a transcatheter heart valve prosthesis protrudes into a ventricle of a native atrioventricular valve.

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

### BRIEF SUMMARY

Embodiments hereof relate to a transcatheter heart valve prosthesis which includes a valve support, a prosthetic valve mounted within the valve support, and an anchoring frame at least partially surrounding the valve support. The anchoring frame and the valve support are attached to each other at respective inflow ends thereof. The anchoring frame includes a fixation portion configured to securely fix the anchoring frame to tissue at a native heart valve, an integration region configured to integrate the anchoring frame with the valve support, and a lateral portion extending between the fixation portion and the integration region. A brim is coupled to and extends radially outwardly from the anchoring frame at a transition between the lateral portion and the fixation portion.

According to another embodiment hereof, a transcatheter heart valve prosthesis includes a valve support, a prosthetic valve mounted within the valve support, and an anchoring frame at least partially surrounding the valve support. The anchoring frame and the valve support are attached to each other at respective outflow ends thereof. The anchoring frame includes a fixation portion configured to securely fix the anchoring frame to tissue at a native heart valve, an integration region configured to integrate the anchoring frame with the valve support, and a lateral portion extending between the fixation portion and the integration region. A brim is coupled to and extends radially outwardly from the fixation portion of the anchoring frame at a brim attachment location. A sealing zone of the fixation portion extends between an inflow end of the anchoring frame and the brim attachment location. At least two rows of barbs are disposed on an outer surface of the anchoring frame along the fixation portion, each barb extending radially outward and towards the inflow end of the anchoring frame. A row of safety cleats is disposed on the outer surface of the anchoring frame along the lateral portion, each barb extending radially outward and generally perpendicular to a central longitudinal axis of the anchoring frame.

### BRIEF DESCRIPTION OF DRAWINGS

FIG. 1 is a perspective view of a transcatheter heart valve prosthesis according to an embodiment hereof, wherein the transcatheter heart valve prosthesis includes a valve support and an anchoring frame which are attached at an inflow end of the transcatheter heart valve prosthesis.

FIG. 1A is a perspective view of a transcatheter heart valve prosthesis according to an embodiment hereof, wherein the transcatheter heart valve prosthesis includes a valve support and an anchoring frame which are attached at an inflow end of the transcatheter heart valve prosthesis, wherein a skirt attached to the transcatheter heart valve prosthesis does not extend to a downstream edge thereof.

FIG. 2 is another perspective view of the transcatheter heart valve prosthesis of FIG. 1, illustrating the outflow end of the transcatheter heart valve prosthesis.

FIG. 3 is another perspective view of the transcatheter heart valve prosthesis of FIG. 1, illustrating the outflow end of the transcatheter heart valve prosthesis.

FIG. 4 is another perspective view of the transcatheter heart valve prosthesis of FIG. 1, illustrating the outflow end of the transcatheter heart valve prosthesis.

FIG. 5 is a perspective view of a portion of the valve support of the transcatheter heart valve prosthesis of FIG. 1.

FIG. 6 is another perspective view of the transcatheter heart valve prosthesis of FIG. 1, illustrating the outflow end of the transcatheter heart valve prosthesis.

FIG. 7 is a schematic side view of the transcatheter heart valve prosthesis of FIG. 1, wherein the anchoring frame of the transcatheter heart valve prosthesis includes a ring skirt and a cone skirt.

FIG. 8 is a schematic side view of the transcatheter heart valve prosthesis of FIG. 1, wherein the anchoring frame of the transcatheter heart valve prosthesis includes a single skirt.

FIG. 8A is a schematic top view of the single skirt of FIG. 8, removed from the transcatheter valve prosthesis and laid flat for illustrative purposes only.

FIG. 9 is a perspective view of a transcatheter heart valve prosthesis according to an embodiment hereof, wherein the transcatheter heart valve prosthesis includes a valve support and an anchoring frame which are attached at an outflow end of the transcatheter heart valve prosthesis.

FIG. 10 is another perspective view of the transcatheter heart valve prosthesis of FIG. 9, illustrating the outflow end of the transcatheter heart valve prosthesis.

FIG. 11 is a top perspective view of the transcatheter heart valve prosthesis of FIG. 9, illustrating the inflow end of the transcatheter heart valve prosthesis.

FIG. 12 is another top perspective view of the transcatheter heart valve prosthesis of FIG. 9, illustrating the inflow end of the transcatheter heart valve prosthesis.

FIG. 13 is a schematic perspective view of a portion of the anchoring frame of the transcatheter heart valve prosthesis of FIG. 9.

FIG. 14 illustrates an as-cut image of a portion of the anchoring frame of the transcatheter heart valve prosthesis of FIG. 9.

FIG. 15 is a side view of an anchoring frame for use in the transcatheter heart valve prosthesis of FIG. 1.

FIG. 16 is a top view of the anchoring frame of FIG. 15.

FIG. 17 is another side view of the anchoring frame of FIG. 15.

FIG. 18 is another top view of the anchoring frame of FIG. 15.

FIG. 19 is an enlarged view of a portion of the anchoring frame of FIG. 15.

FIG. 20 is an enlarged view of a portion of the anchoring frame of FIG. 15.

FIG. 21 is an enlarged view of a portion of the anchoring frame of FIG. 15.

FIG. 22 is a side view of an anchoring frame for use in the transcatheter heart valve prosthesis of FIG. 1.

FIG. 23 is a top view of the anchoring frame of FIG. 22.

FIG. 24 is another side view of the anchoring frame of FIG. 22.

FIG. 25 is another top view of the anchoring frame of FIG. 22.

FIG. 26 is a side perspective view of the anchoring frame of FIG. 22.

FIG. 27 is a side perspective view of an anchoring frame for use in the transcatheter heart valve prosthesis of FIG. 1.

FIG. 28 is a schematic side view of a shape profile of the anchoring frame of FIG. 27.

FIG. 29 is a side perspective view of an anchoring frame for use in the transcatheter heart valve prosthesis of FIG. 1.

FIG. 30 is a schematic top view of the anchoring frame of FIG. 29.

FIG. 31 is a side perspective view of an anchoring frame for use in the transcatheter heart valve prosthesis of FIG. 1.

FIG. 32 is a schematic top view of the anchoring frame of FIG. 31.

FIG. 33 is a side perspective view of an anchoring frame for use in the transcatheter heart valve prosthesis of FIG. 1.

FIG. 34 is a schematic top view of the anchoring frame of FIG. 33.

FIG. 35 is a schematic side view of a transcatheter heart valve prosthesis including the anchoring frame of FIG. 33 positioned at a native heart valve in vivo.

FIG. 36 is a side perspective view of an anchoring frame for use in the transcatheter heart valve prosthesis of FIG. 1.

FIG. 37 is a schematic side view of a shape profile of the anchoring frame of FIG. 36.

FIG. 38A is a schematic side view of a shape profile of an anchoring frame for use in the transcatheter heart valve prosthesis of FIG. 1.

FIG. 38B is a schematic side view of another shape profile of an anchoring frame for use in the transcatheter heart valve prosthesis of FIG. 1.

FIG. 38C is a schematic side view of another shape profile of an anchoring frame for use in the transcatheter heart valve prosthesis of FIG. 1.

### DETAILED DESCRIPTION

Specific embodiments of the present invention are now described with reference to the figures, wherein like reference numbers indicate identical or functionally similar elements. The terms “distal” and “proximal”, when used in the following description to refer to a native vessel, native valve, or a device to be implanted into a native vessel or native valve, such as a heart valve prosthesis, are with reference to the direction of blood flow. Thus, “distal” and “distally” refer to positions in a downstream direction with respect to the direction of blood flow and the terms “proximal” and “proximally” refer to positions in an upstream direction with respect to the direction of blood flow:

The following detailed description is merely exemplary in nature and is not intended to limit the invention or the application and uses of the invention. Although the description of embodiments hereof is in the context of the treatment of heart valves such as the pulmonary; aortic, mitral, or tricuspid valve, the invention may also be used in any other body passageways where it is deemed useful. Furthermore, there is no intention to be bound by any expressed or implied theory presented in the preceding technical field, background, brief summary or the following detailed description.

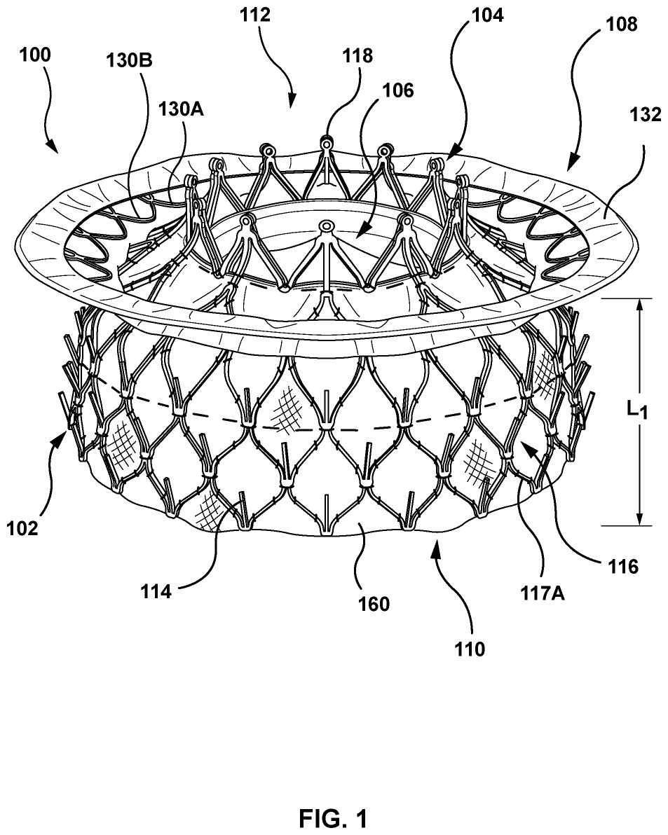

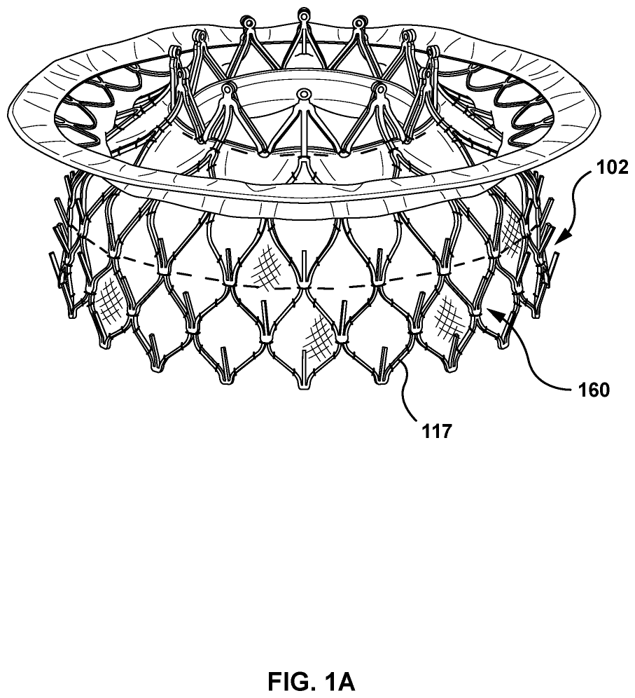

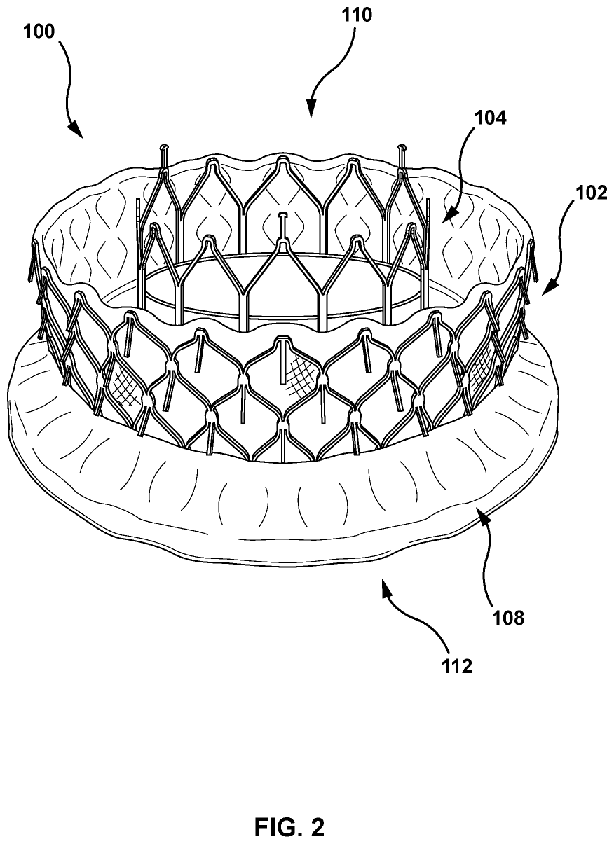

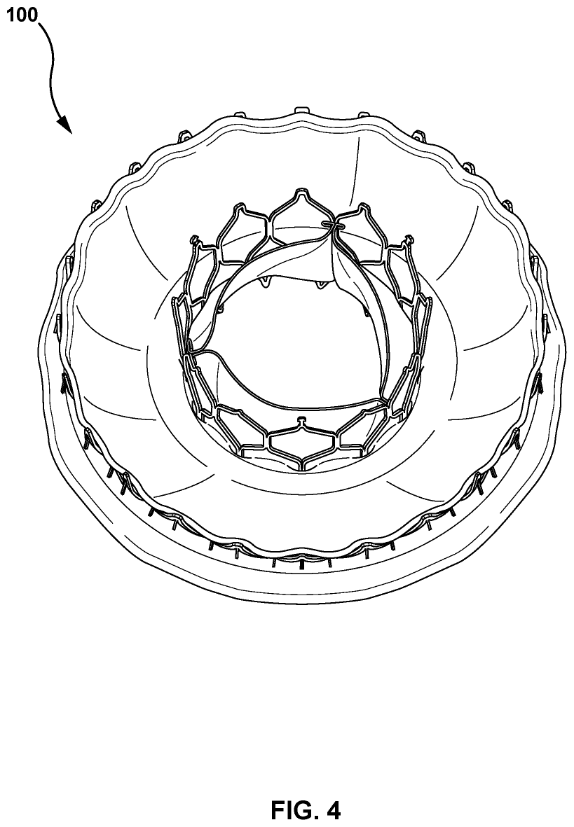

FIGS. 1-8 show an embodiment of a transcatheter heart valve prosthesis 100 according to aspects of the present disclosure. In the embodiment shown, the transcatheter heart valve prosthesis 100 includes an anchoring member or frame 102 at least partially surrounding and coupled to a valve frame or support 104. The transcatheter heart valve prosthesis 100 further includes a prosthetic valve 106 coupled to, mounted within, or otherwise carried by the valve support 104. The transcatheter heart valve prosthesis 100 further includes a brim 108 coupled to and extending radially outwardly from the anchoring frame 102. The transcatheter heart valve prosthesis 100 is configured for placement within a native atrioventricular valve and includes a downstream or distal end, referred to herein as an outflow end 110, and an upstream or proximal end, referred to herein as an inflow end 112. As can be seen, FIG. 1 shows the transcatheter heart valve prosthesis 100 with the inflow end 112 at the top of the figure and the outflow end 110 at the bottom of the figure, and FIG. 2 shows the transcatheter heart valve prosthesis 100 with the outflow end 110 at the top of the figure and the inflow end 112 at the bottom of the figure.

The anchoring frame 102 is a generally tubular component or stent. In the embodiment shown in FIGS. 1-8, the anchoring frame 102 has a funnel-like or hyperboloid shape or profile. Further, the anchoring frame 102 includes openings 116 that may be diamond-shaped. The anchoring frame 102 may be formed by a laser-cut manufacturing method and/or another conventional frame forming methods. For example, the anchoring frame 102 may be laser cut from a single metal tube into the desired geometry, creating a tubular scaffold of interconnected struts 117A that form the openings 116. The anchoring frame 102 may then be shaped into a desired configuration, e.g., funnel-like or hyperboloid shape, using known shape-setting techniques for such materials. It will be understood the anchoring frame 102 may have other shapes and configurations.

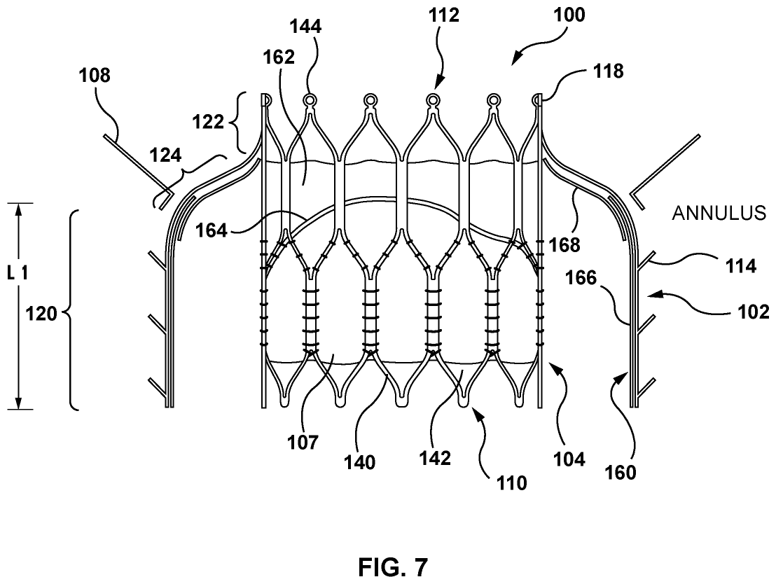

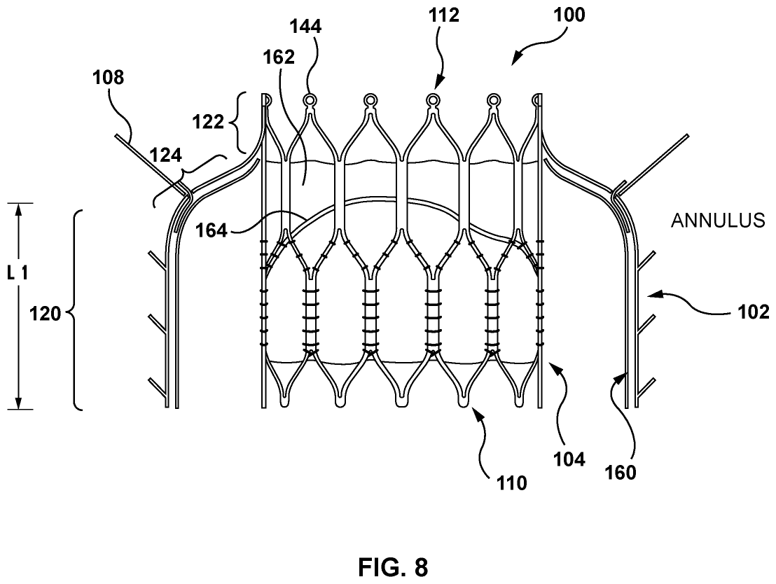

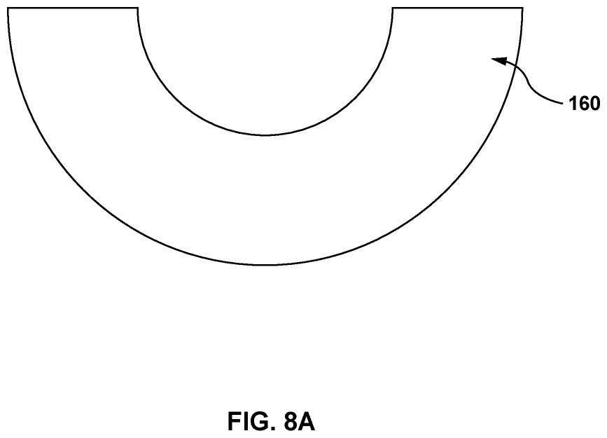

The anchoring frame 102 may include a fixation portion 120 configured to securely fix the anchoring frame 102 to tissue at a native heart valve, an integration region 122 configured to integrate the anchoring frame 102 with the valve support 104, and a lateral portion 124 between the fixation portion 120 and the integration region 122, as best shown in FIGS. 7 and 8.

The anchoring frame 102 may further include tissue engaging elements 114. For example, the tissue engaging elements 114 may be spikes or barbs disposed on an outer wall or surface of the anchoring frame 102 and extending in an upward (towards the inflow end 112) and/or radially outward direction to engage, and in some embodiments, penetrate the native tissue to facilitate retention or maintain position of the transcatheter heart valve prosthesis 100 in a desired implanted location. The anchoring frame 102 may further include a plurality of eyelets 118 disposed at the proximal/inflow end of the anchoring frame 102.

The brim 108 of the transcatheter heart valve prosthesis 100 is coupled to the anchoring frame 102 at the transition between the lateral portion 124 and the fixation portion 120, as best shown in FIGS. 1, 7, and 8. As such, the brim 108 is closer to the inflow end 112 of the transcatheter heart valve prosthesis 100 than it is to the outflow end 110. The brim 108 extends radially outwardly and upwardly (towards the inflow end 112). The brim 108 may be disposed at an angle relative to the outer wall or surface of the anchoring frame 102, for example, between 30 and 90 degrees, or between 40 and 50 degrees. In the embodiment shown in FIGS. 1-6, the brim 108 includes two sinusoidal rings 130A, 130B and a sealing component 132 disposed over or covering at least a downstream surface of the sinusoidal rings 130A, 130B. In the embodiment shown, both the downstream surface and the upstream surface of the sinusoidal rings 130A, 130B are covered with the sealing component 132. The sinusoidal rings 130A, 130B are disposed out of phase relative to each other, and may be woven together or may be disposed in an overlapping manner and coupled together. The sealing component 132 may be a low-porosity woven fabric, such as polyester, polyethylene terephthalate (PET), or polytetrafluoroethylene (PTFE), or may be a knit or woven polyester, such as a polyester or PTFE knit. Alternatively, the sealing component 132 may be formed from a suitable natural or biological material such as pericardium or another membranous tissue including, but not limited to intestinal submucosa. The brim 108 may be attached to the anchoring frame 102 in any suitable manner. In the embodiment shown the sealing component 132 is sewn to the anchoring frame 102 to attach the brim 108 to the anchoring frame 202. The brim 108 serves to locate the transcatheter heart valve prosthesis 100 in the native heart valve. In particular, once the brim 108 is radially expanded in the native heart valve (i.e., upon “flowering” of the brim), the brim 108 is aligned with the upstream surface of the annulus of the native heart valve. Upon alignment of the brim 108 with the upstream surface of the annulus, proper placement of the transcatheter heart valve prosthesis 100 has been achieved, and the transcatheter heart valve prosthesis 100 may be fully deployed and released into the native anatomy.

In the embodiment of FIGS. 1-8, the brim 108 is located a distance or length L1 from the outflow edge of the anchoring frame 102 to the location where the brim 108 is attached to the anchoring frame 102, the length L1 being measured parallel to a central longitudinal axis of the transcatheter heart valve prosthesis 100. The length L1 may be in the range of about 8 mm to about 12 mm.

The valve support 104 may be a generally tubular component or stent that supports the prosthetic valve 106 within the interior of the valve support 104. The valve support 104 is a tubular stent defined by struts 140 defining cells 142. In the embodiment shown, the valve support 104 includes two rows of hexagonal cells 142. The struts 140 defining the distal portion of the proximal row of cells are also the proximal struts 140 defining the distal row of cells. Also in the embodiment shown, the proximal/inflow end of the valve support includes a plurality of eyelets 144. The valve support 104 may be laser cut from a single metal tube into the desired geometry, creating a tubular scaffold of interconnected struts, or may be formed using other methods known to those skilled in the art.

The anchoring frame 102 and the valve support 104 are attached to each other at respective proximal/inflow ends thereof. In the embodiment shown, the respective eyelets 118, 144 of the anchoring frame 102 and the valve support 104 are aligned and attached via rivets. However, in other embodiments, threads, adhesives, solder, laser welding, metal bolts or other mechanical features, or other types of fasteners can be used to secure the valve support 104 to the anchoring frame 104. Further, as shown, the downstream end of the anchoring frame 102 is spaced from the valve support. Thus, forces from the native anatomy during operation of the native valve after the transcatheter heart valve prosthesis 100 has been implanted are mostly absorbed by the anchoring frame 102 such that the valve support 104 and the prosthetic valve 106 coupled thereto remain substantially undeformed. In other words, the valve support 104 and prosthetic valve 106 are effectively mechanically isolated from the distorting forces exerted on the anchoring frame 102 by the native tissue, e.g., radially compressive forces exerted by the native annulus and/or leaflets, longitudinal diastolic and systolic forces, hoop stress, etc.

In embodiments hereof, both the anchoring frame 102 and the valve support 104 are self-expanding to return to a radially expanded state from a radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. “Self-expanding” as used herein means that a structure/component has a mechanical memory to return to the radially expanded configuration or state as described herein. Thus, the transcatheter heart valve prosthesis 100 has a radially compressed configuration for delivery within a delivery system and the radially expanded configuration for deployment within an annulus of the native heart valve site.

The prosthetic valve 106 is coupled within an interior of the valve support 104. The prosthetic valve 106 is configured as a one-way valve to allow blood flow in one direction (i.e., towards the outflow end 110) and thereby regulate blood flow therethrough. The prosthetic valve 106 is capable of blocking flow in one direction to regulate flow therethrough via valve leaflets 107 that may form a bicuspid or tricuspid replacement valve. In the embodiment shown, the leaflets 107 of the prosthetic valve 106 are coupled to the valve support 104 at commissural attachment structures 150 at longitudinal struts of the valve support 104. In the embodiment shown, each row of cell 142 of the valve support includes 12 hexagonal cells. Accordingly, there are 12 longitudinal struts. The commissural attachment structures 150 for the tricuspid prosthetic valve 106 are disposed every fourth longitudinal strut. In other words, there are three longitudinal struts disposed circumferentially between adjacent commissural attachment structures 150. However, this is not meant to be limiting as there may be more or fewer cells around a circumference of the valve support 104. Further, the cells 142 need not be hexagonal, and the prosthetic valve 106 may be attached to other valve supports 104 as known to those skilled in the art. The prosthetic valve 106 may be sutured, riveted, glued, bonded, mechanically interlocked, or otherwise fastened to the struts 140 and/or commissural attachment structures 150.

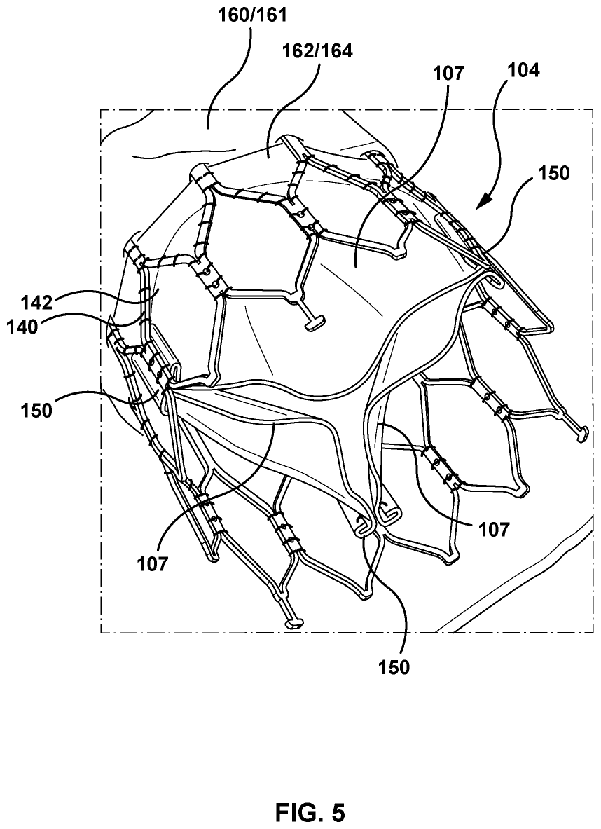

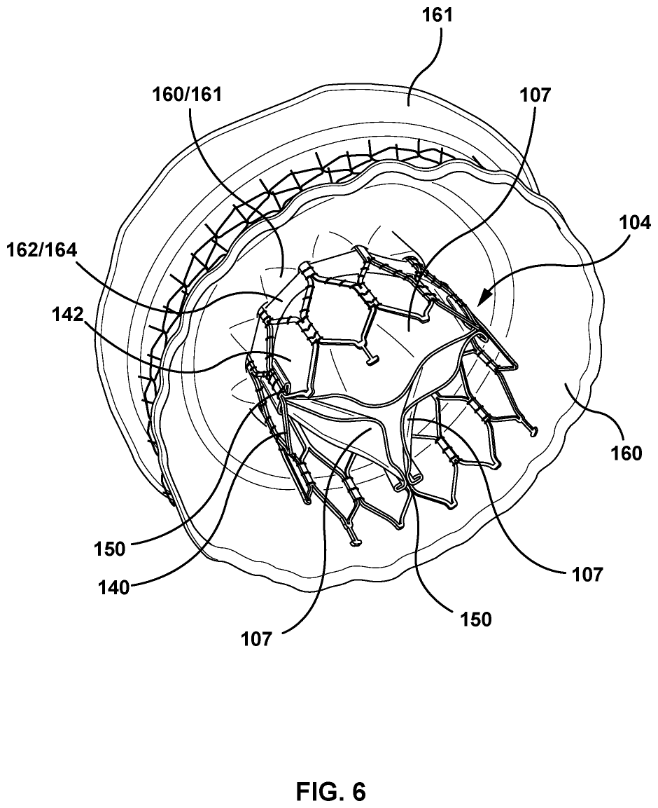

The transcatheter heart valve prosthesis 100 may also include one or more sealing members or skirts coupled to the inner and/or outer surfaces of the anchoring frame 102 and or the valve support 104. In the embodiment shown, the transcatheter heart valve prosthesis 100 includes a first skirt 160 coupled to an inner surface of the anchoring frame 102 and a second skirt 162 coupled to an inner surface of the valve support 104. As shown in FIGS. 5 and 6, a proximal portion 161 of the first skirt 160 is attached to an outer surface of the valve support 104. In other words, the first skirt 160 runs along the inner surface of the anchoring frame 102, folds where the anchoring frame 102 meets the valve support 104, and the proximal portion 161 is attached to the outer surface of the valve support 104.

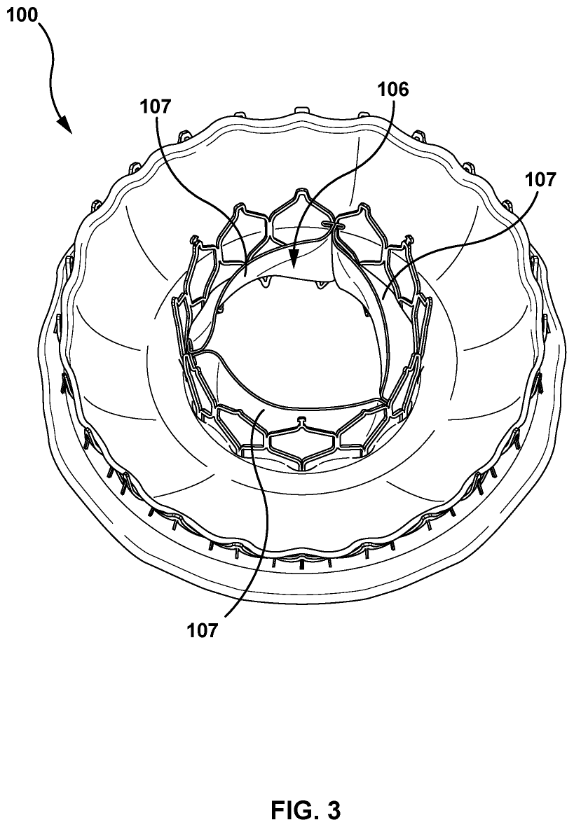

As noted above, the second skirt 162 is attached to the inner surface of the valve support 104. As best shown in FIGS. 3-8, the second skirt 162 is scalloped such that a majority of the inner surface of the valve support is not covered by the second skirt 162. Such a design is enables by the attachment of the anchoring frame 102 to the valve support 104 at the inflow end 112 of the transcatheter heart valve prosthesis 100, which increases leaflet washout and reduced potential thrombus on the leaflets 107 relative to other designs. The scalloped second skirt 162 also provides a lower profile (i.e., smaller diameter) of the transcatheter heart valve prosthesis 100 in the radially compressed configuration by reducing packing density. Further, in other embodiments, the amount of frame material of the valve support 104 may be reduced due to the scalloped second skirt 162 by reducing the need for such frame material to prevent billowing of a non-scalloped second skirt. As shown in FIGS. 3-8, the second skirt 162 is attached to the leaflets 107 along a joint line 164. In particular, a scalloped outflow end of the second skirt 162 is attached to the inflow ends of the leaflets 107 to form the joint line 164. The second skirt 162 may be sutured, riveted, glued, bonded, mechanically interlocked, or otherwise fastened to the leaflets 107. In the embodiment shown, the second skirt is sutured to the leaflets 107 to form the joint line 164.

FIG. 1A shows another embodiment wherein the first skirt 160 does not extend to the downstream end of the anchoring frame 102. Instead, in the embodiment shown in FIG. 1A, the first skirt 160 extends to, but not beyond, the row of struts 117 of the anchoring frame 102 defining the downstream edge of the anchoring frame 102. In other words, the downstream edge of the first skirt 160 is scalloped shaped. A scalloped shaped downstream edge of the first skirt 160 as shown in FIG. 1A may enable more flow into the LVOT as compared to the first 160 as shown in FIG. 1.

The first skirt 160 and the second skirt 162 may be sutured, riveted, glued, bonded, mechanically interlocked, or otherwise fastened to the anchoring frame 102 and the valve support 104, respectively. In the embodiment shown, the first skirt 160 and the second skirt 162 are sutured to the anchoring frame 102 and the valve support 104, respectively. The first skirt 160 and the second skirt 162 are configured to prevent paravalvular leaks between the transcatheter heart valve prosthesis 100 and the native tissue and/or between the anchoring frame 102 and the valve support 104. The first skirt 160 and the second skirt 162 may be formed from a low-porosity woven fabric, such as polyester, polyethylene terephthalate (PET), or polytetrafluoroethylene (PTFE), which creates a one-way fluid passage when attached to the frame. In one embodiment, the first skirt 160 and the second skirt 162 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt 160 and the second skirt 162 may be formed from a suitable natural or biological graft material such as pericardium or another membranous tissue including, but not limited to intestinal submucosa.

In an embodiment, as shown in FIG. 7, the first skirt 160 may include two skirts. In particular, the first skirt 160 may include a ring skirt 166 and a cone skirt 168. The ring skirt 166 may be generally cylindrical. In another embodiment, as shown in FIG. 8, the first skirt 160 may be a single piece skirt, as shown in FIG. 8A. Further details of single piece first skirts 160 for anchoring frames 102 may be found in U.S. Provisional Application No. 63/267,262, filed Jan. 28, 2022, which is incorporated by reference herein in its entirety.

As discussed in the Background section above, it is desirable for a transcatheter heart valve prosthesis implanted at a native mitral valve to not obstruct the left ventricular outflow track (LVOT). With respect to the transcatheter heart valve prosthesis 100, as noted above, the outflow end of the anchoring frame 102 is not attached to the valve support 104. As such, as explained above, the anchoring frame 102 absorbs forces from the native anatomy. Due to the cantilever-like connection of the anchoring frame 102 to the valve support 104, the downstream end of the fixation portion 120 of the anchoring frame 102 bends further radially inward than the upstream end of the fixation portion 120 where the fixation portion 120 meets the lateral portion 124. Thus, once implanted the fixation portion 120 of the anchoring frame 102 is angled radially inwardly towards the valve support 104 due to forces acting on the anchoring frame 102. This radially inward inclination provides more area in the LVOT than a similarly implanted valve without the radially inward inclination.

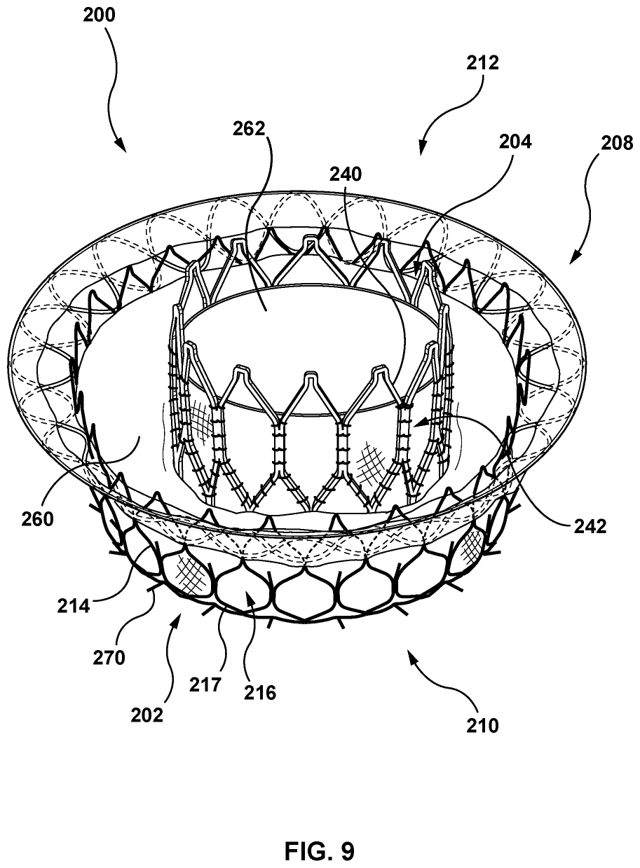

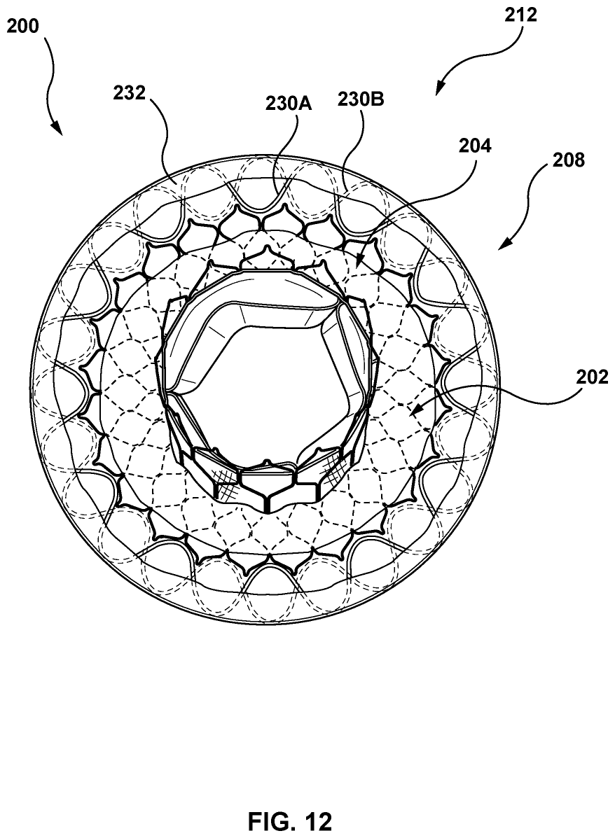

FIGS. 9-14 show an embodiment of a transcatheter heart valve prosthesis 200 according to aspects of the present disclosure. In the embodiment shown, the transcatheter heart valve prosthesis 200 includes an anchoring member or frame 202 at least partially surrounding and coupled to a valve frame or support 204. The transcatheter heart valve prosthesis 200 further includes a prosthetic valve 206 coupled to, mounted within, or otherwise carried by the valve support 204. The transcatheter heart valve prosthesis 200 further includes a brim 208 coupled to and extending radially outwardly from the anchoring frame 202. The transcatheter heart valve prosthesis 200 is configured for placement within a native atrioventricular valve and includes a downstream or distal end, referred to herein as an outflow end 210, and an upstream or proximal end, referred to herein as an inflow end 212.

The anchoring frame 202 is a generally tubular component or stent. In the embodiment shown in FIGS. 9-14, the anchoring frame 202 has a funnel-like or hyperboloid shape or profile. Further, the anchoring frame 202 includes openings or cells 216 that may be diamond-shaped. The anchoring frame 202 may be formed by a laser-cut manufacturing method and/or another conventional frame forming methods. For example, the anchoring frame 202 may be laser cut from a single metal tube into the desired geometry, creating a tubular scaffold of interconnected struts 217 that form the cells 216. The anchoring frame 202 may then be shaped into a desired configuration, e.g., funnel-like or hyperboloid shape, using known shape-setting techniques for such materials. It will be understood the anchoring frame 202 may have other shapes and configurations.

The anchoring frame 202 may include a fixation portion 220 configured to securely fix the anchoring frame 202 to tissue at a native heart valve, an integration region 222 configured to integrate the anchoring frame 202 with the valve support 204, and a lateral portion 224 between the fixation portion 220 and the integration region 222, as best shown in FIG. 13.

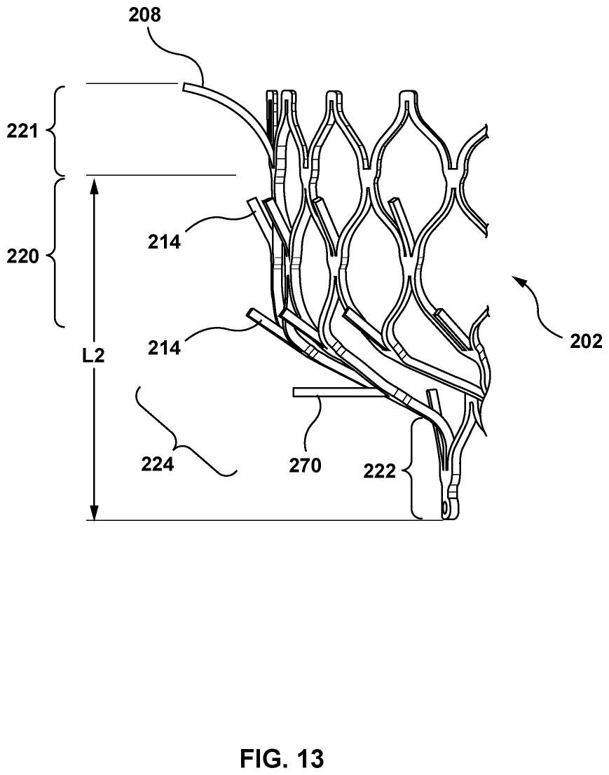

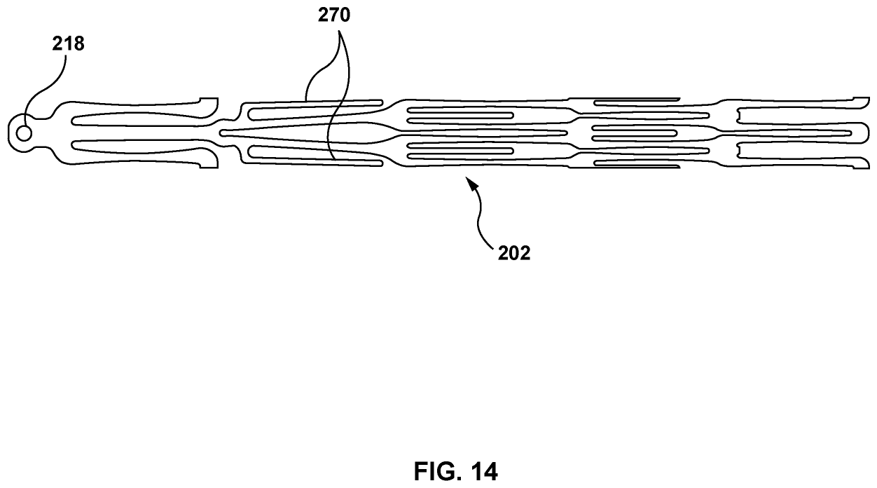

The anchoring frame 202 may further include tissue engaging elements 214. For example, the tissue engaging elements 214 may be spikes or barbs disposed on an outer wall or surface of the anchoring frame 202 and extending in an upward (towards the inflow end 212) and/or radially outward direction to engage, and in some embodiments, penetrate the native tissue to facilitate retention or maintain position of the transcatheter heart valve prosthesis 200 in a desired implanted location. As can be seen in FIG. 13, the anchoring frame 202 does not include barbs 214 proximal of the location where the brim 208 is attached to the anchoring frame 202. The anchoring frame 202 further includes cactus or safety cleats 270 extending radially outwardly from the anchoring frame 202 in the radially expanded configuration. The safety cleats 270 are located in the lateral portion 224 of the anchoring frame. The safety cleats 270 are provided as an additional measure to prevent movement of the transcatheter heart valve prosthesis 200 into the atrium. In particular, although the safety cleats 270 are located in the lateral portion 224 of the anchoring frame 202, native leaflets of the native valve tend to follow the shape of the anchoring frame 202 of the transcatheter heart valve prosthesis 200. Thus, the safety cleats 270 embed into the native leaflets, thereby preventing the transcatheter heart valve prosthesis 200 from being displaced into the atrium. FIG. 14 shows an as-cut image of a portion of the anchoring frame 202 showing the location of two of the safety cleats 270. The other safety cleats 270 may be located similarly around the circumference of the anchoring frame. The anchoring frame 202 may further include a plurality of eyelets 218 disposed at the distal/outflow end of the anchoring frame 202.

The brim 208 of the transcatheter heart valve prosthesis 200 is coupled to the anchoring frame 202 adjacent the inflow end 212 of the transcatheter heart valve prosthesis 200. In particular, as shown in FIG. 13, the brim 208 is attached to the anchoring frame 202 about 5 mm distal of the inflow edge of the anchoring frame 202. The brim 208 may be attached to the anchoring frame 202 from about 4 mm to about 10 mm from the inflow edge of the anchoring frame 202. In other embodiments, the brim 208 may be attached to the anchoring frame 202 from about 4 mm from the inflow edge of the anchoring frame 202 to a about a location more than 4 mm from the inflow edge of the anchoring frame 202 such that there is sufficient fixation distal of the brim attachment location. As such, the brim 208 is closer to the inflow end 212 of the transcatheter heart valve prosthesis 100 than it is to the outflow end 210. Put another way, in the embodiment of FIGS. 9-14, the brim 208 is located a distance or length L2 from the outflow edge of the anchoring frame 202 to the location where the brim 208 is attached to the anchoring frame 202, the length L2 being measured parallel to a central longitudinal axis of the transcatheter heart valve prosthesis 200. In the embodiment shown, the length L2 may be about 12 mm to about 18 mm.

The brim 208 extends radially outwardly and upwardly (towards the inflow end 212). The brim 208 may be disposed at an angle relative to the outer wall or surface of the anchoring frame 202, for example, between 30 and 90 degrees, or between 40 and 50 degrees. It is estimated that as compared to a similar transcatheter heart valve prosthesis with a brim attached at the inflow edge of the anchoring frame, the transcatheter heart valve prosthesis 200 of FIGS. 9-14 with the brim 208 attached about 5 mm distal of the inflow edge of the anchoring frame 202 results in a 15% increase in patients eligible to receive such a transcatheter heart valve prosthesis due increased flow (i.e., reduced obstruction) through in the LVOT. In other words, patients that would not be eligible to receive a transcatheter heart valve prosthesis due to the heart valve prosthesis obstruction the LVOT may receive the transcatheter heart valve prosthesis 200.

In the embodiment shown in FIGS. 9-14, the brim 208 includes two sinusoidal rings 230A, 230B and a sealing component 232 disposed over or covering at least a downstream surface of the sinusoidal rings 230A, 230B. In the embodiment shown, both the downstream surface and the upstream surface of the sinusoidal rings 230A, 230B are covered with the sealing component 232. The sinusoidal rings 230A, 230B are disposed out of phase relative to each other, and may be woven together or may be disposed in an overlapping manner and coupled together. The sealing component 232 may be a low-porosity woven fabric, such as polyester, polyethylene terephthalate (PET), or polytetrafluoroethylene (PTFE), or may be a knit or woven polyester, such as a polyester or PTFE knit. Alternatively, the sealing component 232 may be formed from a suitable natural or biological material such as pericardium or another membranous tissue including, but not limited to intestinal submucosa.

The brim 208 may be attached to the anchoring frame 202 in any suitable manner. In the embodiment shown the sealing component 232 is sewn to the anchoring frame 202 to attach the brim 208 to the anchoring frame 202. The brim 208 serves to locate the transcatheter heart valve prosthesis 200 in the native heart valve. In particular, once the brim 208 is radially expanded in the native heart valve (i.e., upon “flowering” of the brim), the brim 208 is aligned with the upstream surface of the annulus of the native heart valve. Upon alignment of the brim 208 with the upstream surface of the annulus, proper placement of the transcatheter heart valve prosthesis 200 has been achieved, and the transcatheter heart valve prosthesis 200 may be fully deployed and released into the native anatomy.

With the brim 208 attached to the anchoring frame 202 distal of the inflow edge of the anchoring frame 202, the brim 208 may act to prevent paravalvular leakage. In embodiments with the brim attached at the inflow edge of the anchoring frame, the brim sits above the annulus of the native valve. With the brim in such embodiments being attached to the anchoring frame via suturing the sealing member to the anchoring frame, the brim does not prevent paravalvular leakage because it is too flimsy to prevent blood blow. With the brim 208 of the transcatheter heart valve prosthesis 200 shifted distally, the brim 208 about against tissue of the native valve and the anchoring frame 202 acts as a backstop to the brim 208. Thus, as shown in FIG. 13, the portion of the anchoring frame 202 above where the brim 208 is attached to the anchoring frame 202 acts as a seal zone 221. Further, the fixation portion 220, which includes a skirt described below, is a fixation and seal zone. The lateral portion 224 including the safety cleat 270 may act as a fixation zone.

The valve support 204 may be a generally tubular component or stent that supports the prosthetic valve 206 within the interior of the valve support 206. The valve support 204 is a tubular stent defined struts 240 defining cells 242. In the embodiment shown, the valve support 204 includes two rows of hexagonal cells 242. The struts 240 defining the distal portion of the proximal row of cells are also the proximal struts 240 defining the distal row of cells. Also in the embodiment shown, the distal/outflow end of the valve support 204 includes a plurality of eyelets 244. The valve support 204 may be laser cut from a single metal tube into the desired geometry, creating a tubular scaffold of interconnected struts, or may be formed using other methods known to those skilled in the art.

The anchoring frame 202 and the valve support 204 are attached to each other at respective distal/outflow ends thereof. In the embodiment shown, the respective eyelets 218, 244 of the anchoring frame 202 and the valve support 204 are aligned and attached via rivets. However, in other embodiments, threads, adhesives, solder, laser welding, metal bolts or other mechanical features, or other types of fasteners can be used to secure the valve support 202 to the anchoring frame 204.

In embodiments hereof, both the anchoring frame 202 and the valve support 204 are self-expanding to return to a radially expanded state from a radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. “Self-expanding” as used herein means that a structure/component has a mechanical memory to return to the radially expanded configuration or state as described herein. Thus, the transcatheter heart valve prosthesis 100 has a radially compressed configuration for delivery within a delivery system and the radially expanded configuration for deployment within an annulus of the native heart valve site.

The prosthetic valve 206 is coupled within an interior of the valve support 204. The prosthetic valve 206 is configured as a one-way valve to allow blood flow in one direction (i.e., towards the outflow end 210) and thereby regulate blood flow therethrough. The prosthetic valve 206 is capable of blocking flow in one direction to regulate flow therethrough via valve leaflets 207 that may form a bicuspid or tricuspid replacement valve. In the embodiment shown, the leaflets 207 of the prosthetic valve 106 are coupled to the valve support 204 at commissural attachment structures at longitudinal struts of the valve support 204. In the embodiment shown, each row of cells 242 of the valve support 204 includes 12 hexagonal cells. Accordingly, there are 12 longitudinal struts. The commissural attachment structures for the tricuspid prosthetic valve 206 are disposed every fourth longitudinal strut. In other words, there are three longitudinal struts disposed circumferentially between adjacent commissural attachment structures. However, this is not meant to be limiting as there may be more or fewer cells around a circumference of the valve support 204. Further, the cells 242 need not be hexagonal, and the prosthetic valve 104 may be attached to other valve supports 204 as known to those skilled in the art. The prosthetic valve 206 may be sutured, riveted, glued, bonded, mechanically interlocked, or otherwise fastened to the struts and/or commissural attachment structures.

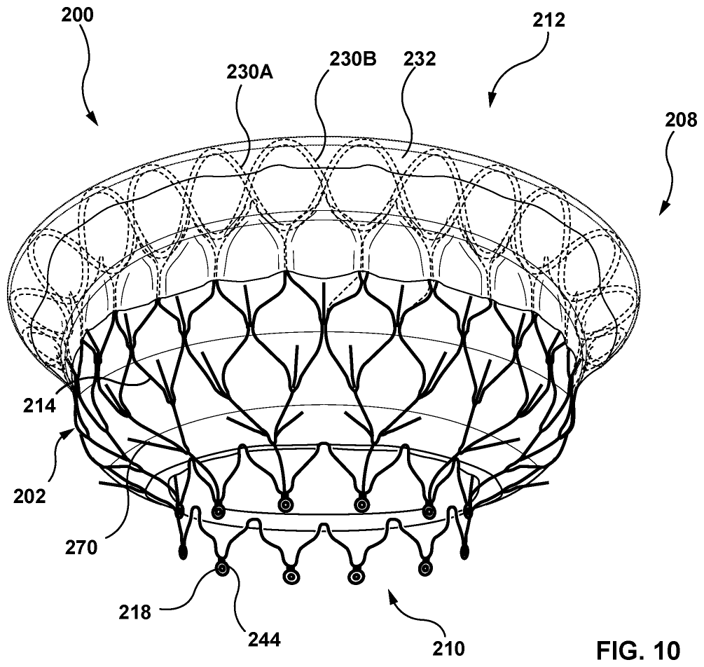

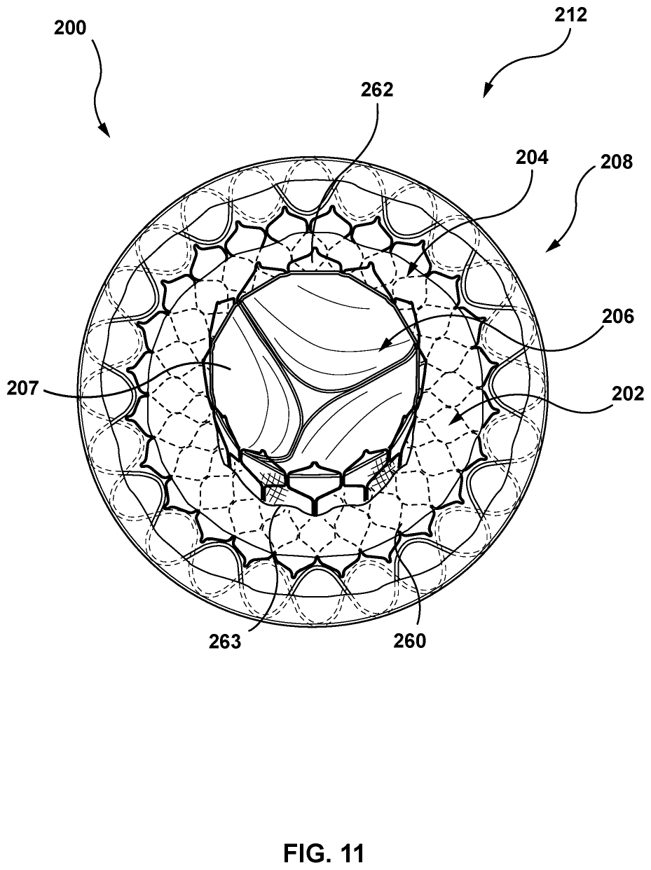

The transcatheter heart valve prosthesis 200 may also include one or more sealing members or skirts coupled to the inner and/or outer surfaces of the anchoring frame 202 and or the valve support 204. In the embodiment shown, the transcatheter heart valve prosthesis 200 includes a first skirt 260 coupled to an inner surface of the anchoring frame 202 and a second skirt 262 coupled to an inner surface of the valve support 104. As shown in FIG. 11, a distal portion 263 of the first skirt 260 is attached to an outer surface of the valve support 204. In other words, the first skirt 260 runs along the inner surface of the anchoring frame 202, folds where the anchoring frame 202 meets the valve support 104, and the distal portion 263 is attached to the outer surface of the valve support 204.

As noted above, the second skirt 262 is attached to the inner surface of the valve support 204. As best shown in FIGS. 9, 11, and 12, the second skirt 262 extends the majority of the length of the inner surface of the valve support 204. However, this is not limiting, and the extent of the second skirt 262 may be selected as needed for proper functioning of the valve. Further, the leaflets 207 of the prosthetic valve 206 are attached to the second skirt 262 at a proximal end of the leaflets 207 along a joint line. The second skirt 262 may be sutured, riveted, glued, bonded, mechanically interlocked, or otherwise fastened to the leaflets 207. In the embodiment shown, the second skirt is sutured to the leaflets 207.

The first skirt 260 and the second skirt 262 may be sutured, riveted, glued, bonded, mechanically interlocked, or otherwise fastened to the anchoring frame 202 and the valve support 204, respectively. In the embodiment shown, the first skirt 260 and the second skirt 262 are sutured to the anchoring frame 202 and the valve support 204, respectively. The first skirt 260 and the second skirt 262 are configured to prevent paravalvular leaks between the transcatheter heart valve prosthesis 200 and the native tissue and/or between the anchoring frame 202 and the valve support 204. The first skirt 260 and the second skirt 262 may be formed from a low-porosity woven fabric, such as polyester, polyethylene terephthalate (PET), or polytetrafluoroethylene (PTFE), which creates a one-way fluid passage when attached to the frame. In one embodiment, the first skirt 260 and the second skirt 262 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt 160 and the second skirt 162 may be formed from a suitable natural or biological graft material such as pericardium or another membranous tissue including, but not limited to intestinal submucosa.

As described above with respect to the first skirt 160 of the transcatheter heart valve prosthesis 100, the first skirt 260 of the transcatheter heart valve prosthesis 200 may include two skirts (a ring skirt and a cone skirt), or may be a single piece skirt. Further details of single piece first skirts for anchoring frames may be found in U.S. Provisional Application No. 63/267,262, filed Jan. 28, 2022, which is incorporated by reference herein in its entirety.

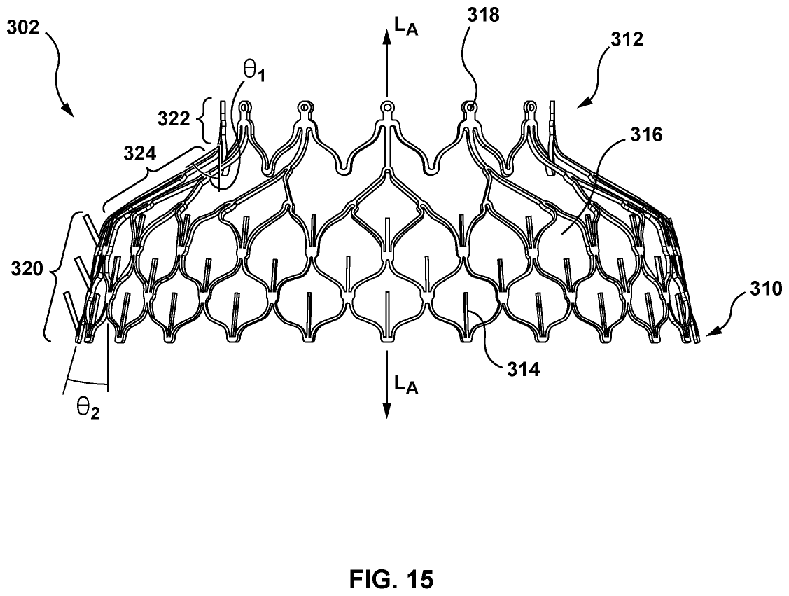

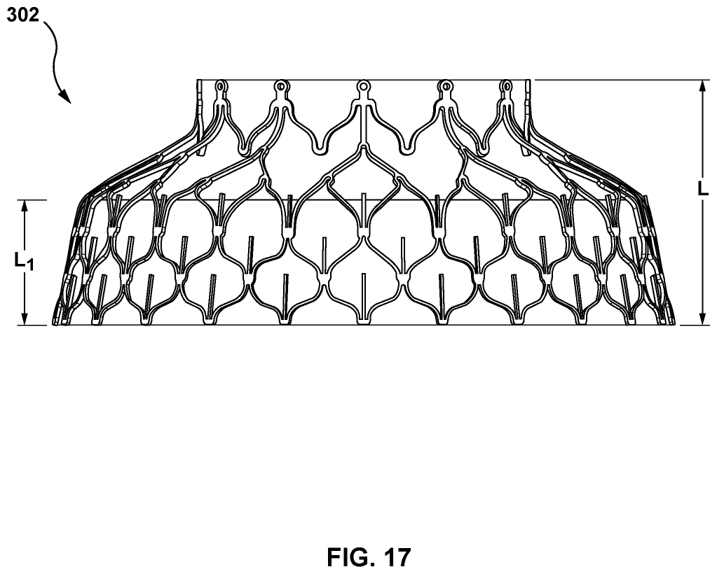

As stated above, the anchoring frame 102 may have other shapes and configurations other than the shape depicted in FIGS. 1-8. FIGS. 15-21 show an embodiment of an anchoring member or frame 302 for use in the transcatheter heart valve prosthesis 100. The anchoring member or frame 302 is configured to at least partially surround and be coupled to the valve support 104. FIGS. 15-18 show the anchoring frame 302 without the valve support attached, for clarity purposes only. The anchoring frame 302 is configured for placement within a native atrioventricular valve and includes a downstream or distal end, referred to herein as an outflow end 310, and an upstream or proximal end, referred to herein as an inflow end 312. As can be seen, FIG. 15 shows the anchoring frame 302 with the inflow end 312 at the top of the figure and the outflow end 310 at the bottom of the figure. The anchoring frame 302 may have a length L (see FIG. 17), extending from the inflow end 312 to the outflow end 310, of about 20 mm-25 mm, for example. The anchoring frame 302 may include a plurality of eyelets 318 disposed at the proximal/inflow end of the anchoring frame 302, for example.

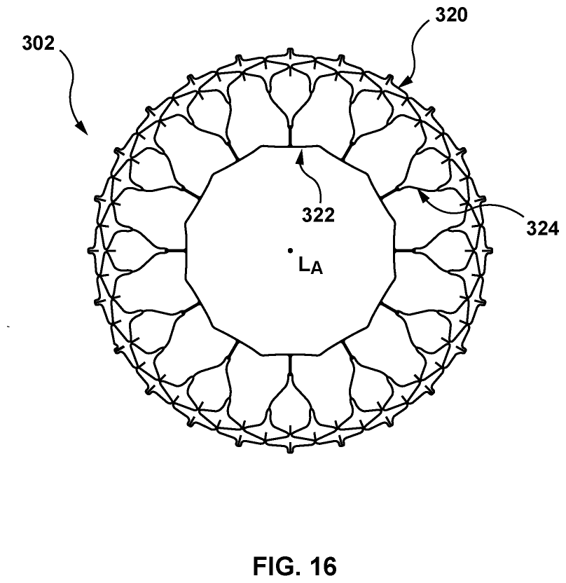

When in a radially expanded configuration, the anchoring frame 302 is configured to secure the heart valve prosthesis to the native valve and the surrounding tissue, such as the inward facing-surface of the native leaflets. FIGS. 15-21 depict the anchoring frame 302 in its radially expanded configuration. The anchoring frame 302 is a generally tubular component or stent and forms a hollow cylindrical shape around a central longitudinal axis LA. In some embodiments, the anchoring frame 302 has a hollow cylindrical shape that resembles a D-shape, similar to the shape of a native mitral valve annulus, for example. In the embodiment shown in FIGS. 15-21, the anchoring frame 302 has a bell shape or profile which includes two flared portions. More particularly, with reference to the side and top views of FIGS. 15 and 16, respectively, the anchoring frame 302 includes a fixation portion 320 configured to securely fix the anchoring frame 302 to tissue at a native heart valve, an integration region 322 configured to integrate the anchoring frame 302 with the valve support, and a lateral portion 324 between the fixation portion 320 and the integration region 322.

The lateral portion 324 is similar to the lateral portion 124, and it extends at an angle Θ₁ relative to the central longitudinal axis LA of the anchoring frame 302. In an embodiment, angle Θ₁ is between 40°-60° relative to the central longitudinal axis LA. In the direction from the inflow end 312 to the outflow end 310, the lateral portion 324 flares or extends radially outward at the angle Θ₁. The lateral portion 324 depicted is a portion of the anchoring frame 302 that is generally conically shaped and extends radially outwardly in the direction of bloodflow therethrough, i.e., from the inflow end 312 to the outflow end 310. The lateral portion 324 may be generally straight, or it may be generally curved with one or more arcs, including S- and U-shapes. In some embodiments, the lateral portion 324 is shape set with curvatures that mate with, mirror, or mimic the shape of a mitral annulus to allow for improved conformity when deployed. The lateral portion 324 may be shape set with curvatures to allow for increased conformity to the native heart-valve anatomy when implanted in vivo. However, this not meant to be limiting, and in other embodiments, the lateral portion 324 may be substantially straight. In other embodiments, the lateral portion 324 may help the anchoring frame 302 conform to the anatomy by being relatively flexible.

As depicted, the fixation portion 320 of the anchoring frame is integrally formed with the lateral portion 324 of the anchoring frame 302. In other embodiments, the fixation portion 320 and the lateral portion 324 are made integral with an attachment mechanism, such as one or more hooks, hinges, or rivets. The fixation portion 320 extends at an angle Θ2 relative to the central longitudinal axis LA of the anchoring frame 302. In an embodiment, angle Θ2 is between 10° and 30° relative to the central longitudinal axis LA. In the direction of bloodflow therethrough, from the inflow end 312 to the outflow end 310, the fixation portion 320 flares or extends radially outward at the angle Θ2. The fixation portion 320 may be generally straight, or it may be generally curved with one or more arcs, including S-, U-, or hourglass shapes. In some embodiments, the fixation portion 320 is shape set with curvatures that mirror or mimic the shape of a mitral annulus to allow for improved conformity when deployed. The fixation portion 320 may be shape set with curvatures, such as S-, U-, or hourglass shapes, to allow for increased conformity to the native heart-valve anatomy when implanted in vivo. However, this not meant to be limiting, and in other embodiments, the fixation portion 320 may be generally straight and may help the anchoring frame 302 conform to the anatomy by being relatively flexible. The fixation portion 320 of the anchoring frame 302 may have a length L1 (see FIG. 17), extending from the outflow end 310 to the junction between the fixation portion 320 and the lateral portion 324, of about 10 mm-14 mm, for example.

The lateral portion 324 and the fixation portion 320 may thus be flared at various angles and collectively provide the anchoring frame 302 with a bell shape or profile. The bell shape or profile is configured to conform to the native annulus of, for example, a native mitral or tricuspid valve. In addition, with the fixation portion 320 flared radially outward rather than parallel to the central longitudinal axis LA, some embodiments reduce the amount of the anchoring frame 302 that protrudes or extends into or around the LVOT. Thus, obstruction of blood flow through the LVOT may be minimized or prevented with such designs.

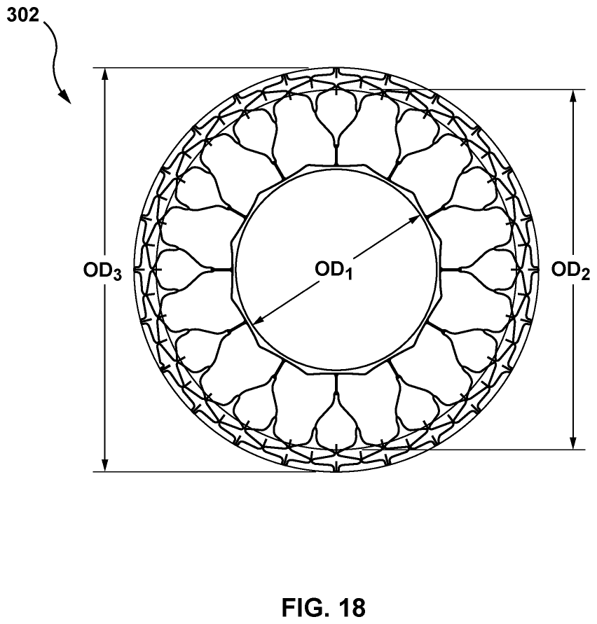

With reference to the side and top views of FIGS. 17 and 18, respectively, the bell shape or profile of the anchoring frame 302 is also apparent via a comparison of the outer diameters along the length L of the anchoring frame 302. More particularly, the anchoring frame 302 has an outer diameter OD₁ at the inflow end 312, an outer diameter OD₂ at the junction between the lateral portion 324 and the fixation portion 320, and an outer diameter OD₃ at the outflow end 310. The outer diameters increase in the direction of the bloodflow therethrough, with the outer diameter OD₂ being greater than the outer diameter OD₁ and the outer diameter OD₃ being greater than the outer diameter OD₂. In an embodiment, the outer diameter OD₁ ranges between 26 mm-30 mm, the outer diameter OD₂ ranges between 45 mm-55 mm, and the outer diameter OD₃ ranges between 50 mm and 60 mm, for example.

The anchoring frame 302 includes openings or cells 316 that may be diamond-shaped. The anchoring frame 302 may be formed by a laser-cut manufacturing method and/or another conventional frame forming methods. For example, the anchoring frame 302 may be laser cut from a single metal tube into the desired geometry, creating a tubular scaffold of interconnected struts that form the openings 316. The anchoring frame 302 may then be shaped into a desired configuration, e.g., bell shape or profile which includes two or more flared portions at varying angles, using known shape-setting techniques for such materials.

In embodiments hereof, the anchoring frame 302 is self-expanding to return to a radially expanded state from a radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. Thus, the anchoring frame 302 has a radially compressed configuration for delivery within a delivery system and the radially expanded configuration for deployment within an annulus of the native heart valve.

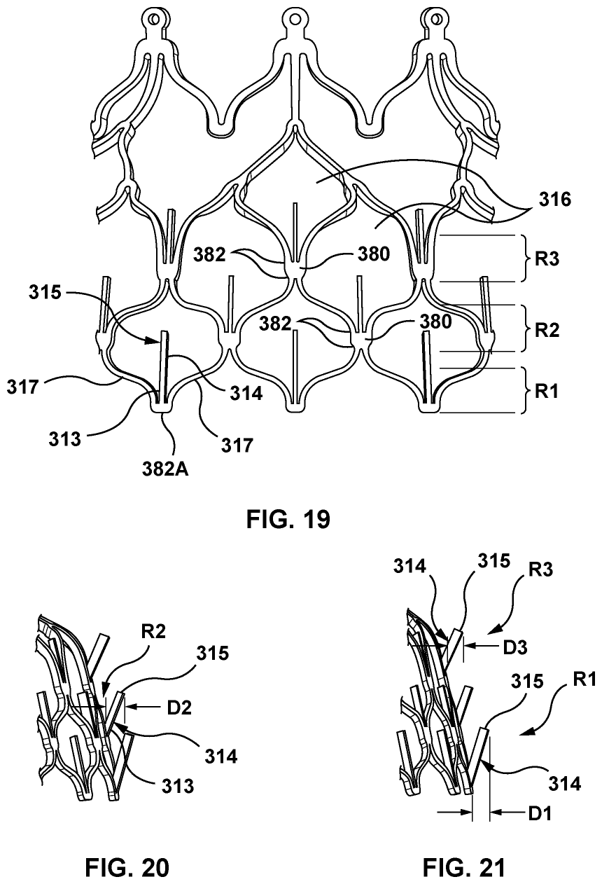

Turning to FIGS. 19-21, the anchoring frame 302 further includes tissue engaging elements 314. The tissue engaging elements 314 may be cleats or barbs disposed on an outer wall or surface of the anchoring frame 302 and extending in an upward (towards the inflow end 312) and/or radially outward direction to engage, and in some embodiments, penetrate the native tissue to facilitate retention or maintain position of the transcatheter heart valve prosthesis in a desired implanted location. In another embodiment, one or more of the tissue engaging elements 314 may extend in a downward (towards the outflow end 310) and/or radially outward direction to engage, and in some embodiments, penetrate the native tissue to facilitate retention or maintain position of the transcatheter heart valve prosthesis in a desired implanted location. In an embodiment, each tissue engaging element 314 has a blunt, atraumatic unattached, or free, end 315.

Each tissue engaging element 314 is disposed at a node 380 of the anchoring frame 302 or at an endmost outflow crown 382A of the anchoring frame 302. More particularly, with reference to FIG. 19, the fixation portion 320 may be considered to include a plurality of crowns 382 and a plurality of struts 317 with each crown 382 being formed between a pair of opposing struts 317. Each crown 382 is a curved segment or bend extending between opposing struts 317. Along the outflow end 310 of the anchoring frame, the crowns are referred to herein as the endmost outflow crowns 382A. The openings or cells 316 of the anchoring frame 302 are defined by edges of the plurality of crowns 382 and the plurality of struts 317. A node 380 is defined as a region where two crowns of the plurality of crowns 382 within the anchoring frame 302 meet or connect.

The fixation portion 320 of the anchoring frame 302 in some embodiments includes three rows R1, R2, R3 of tissue engaging elements 314. Row R1 of the tissue engaging elements 314 is disposed at the outflow end 310 of the anchoring frame 302. Row R2 of the tissue engaging elements 314 is disposed proximal of row R1, and row R3 of the tissue engaging elements 314 is disposed proximal of row R2. FIG. 20 is an enlarged view in a plane normal to the viewer of a tissue engaging element 314 of the row R2. As depicted, the unattached end 315 of each tissue engaging element 314 of the row R2 is disposed a radial distance D2 from a base 313 of the tissue engaging element 314. FIG. 21 is an enlarged view in a plane normal to the viewer of tissue engaging elements 314 of the rows R1, R3. As depicted, the unattached end 315 of each tissue engaging element 314 of the row R1 is disposed a radial distance D₁ from a base 313 of the tissue engaging element 314, and the unattached end 315 of each tissue engaging element 314 of the row R3 is disposed a radial distance D₃ from a base 313 of the tissue engaging element 314. In an embodiment, radial distance D₂ and radial distance D₃ are substantially equal and range between 1.05 mm and 1.15 mm, for example. In an embodiment, radial distance D₃ ranges between 2.85 mm and 3.70 mm, for example.

Although not shown, in some embodiments, the anchoring frame 302 further includes a brim such as brim 108 or brim 208 described above with respect to the anchoring frames 102 and 202, respectively. The brim may be coupled to and extend radially outwardly from the anchoring frame 302. In other embodiments, the anchoring frame 302 does not include a brim.

The anchoring frame 302 may also include one or more sealing members, membranes, or skirts coupled to the inner and/or outer surfaces of the anchoring frame 302. In an embodiment, the anchoring frame 302 includes a first skirt (similar to the first skirt 160) coupled to an inner surface of the anchoring frame 302. The first skirt is configured to prevent paravalvular leaks between the anchoring frame 302 and the native tissue and/or between the anchoring frame 302 and the valve support. The first skirt may be formed from a low-porosity woven fabric, such as polyester, polyethylene terephthalate (PET), or polytetrafluoroethylene (PTFE). The first skirt may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

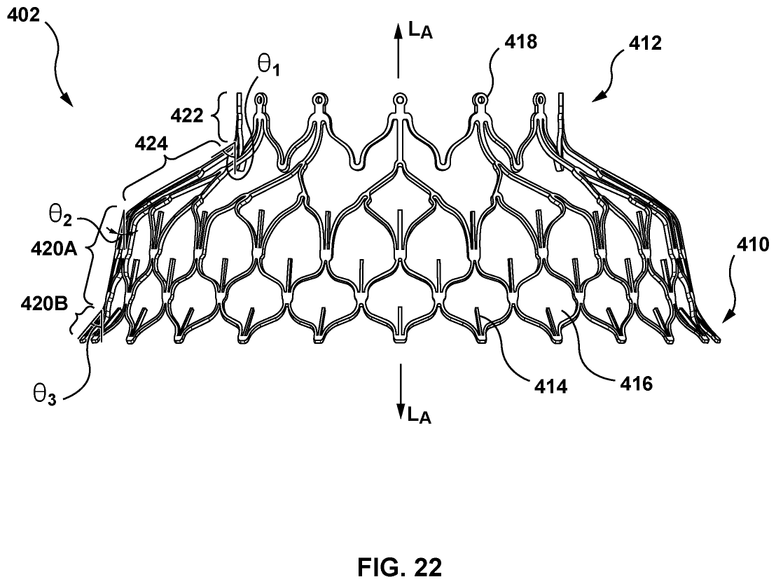

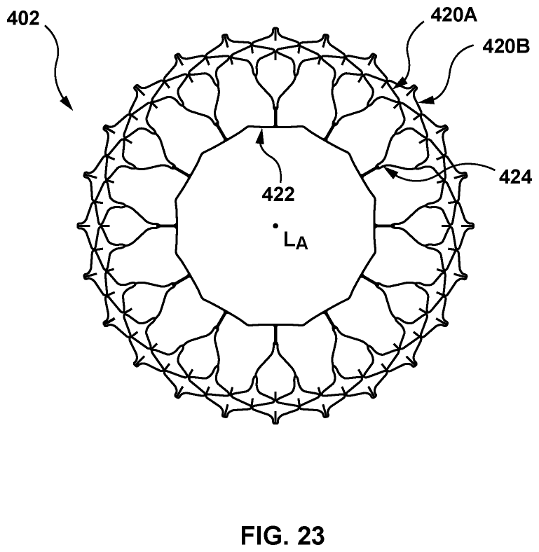

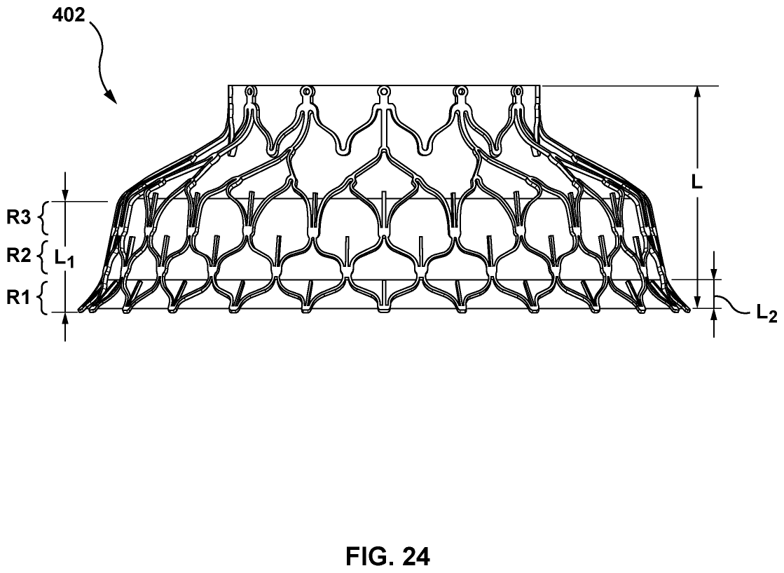

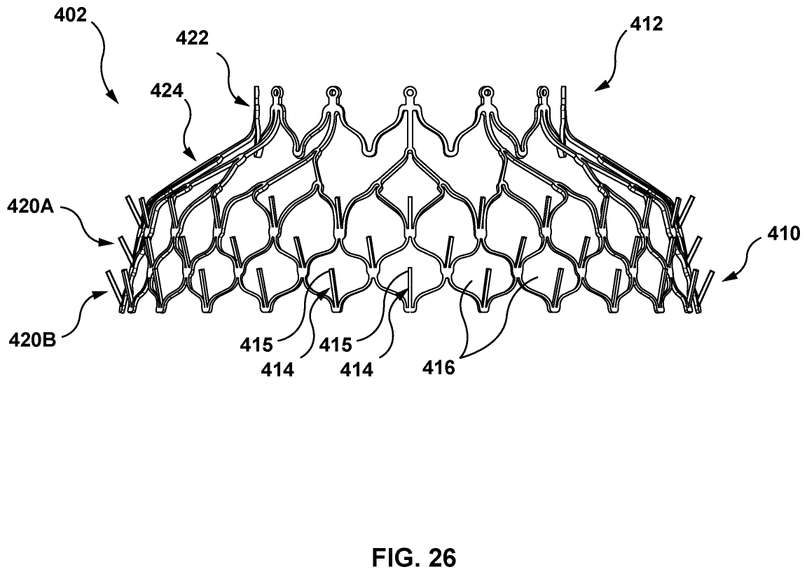

FIGS. 22-26 show another embodiment of an anchoring member or frame 402 for use in the transcatheter heart valve prosthesis 100. The anchoring member or frame 402 is configured to at least partially surround and be coupled to the valve support 104. FIGS. 22-26 show the anchoring frame 402 without the valve support attached, for clarity only. The anchoring frame 402 is configured for placement within native atrioventricular valve anatomy and includes a downstream or distal or outflow end 410, and an upstream or proximal or inflow end 412. As can be seen, FIG. 22 shows the anchoring frame 402 with the inflow end 412 at the top of the figure and the outflow end 410 at the bottom of the figure. The anchoring frame 402 may have a length L (see FIG. 24), extending from the inflow end 412 to the outflow end 410, of about 23 mm-27 mm, for example. The anchoring frame 402 may include a plurality of eyelets 418 disposed at the proximal/inflow end of the anchoring frame 402, for example.

When in a radially expanded configuration, the anchoring frame 402 is configured to secure the prosthesis to the native valve and the surrounding tissue, such as the inward facing-surface of the native leaflets. FIGS. 22-26 depict the anchoring frame 402 in its radially expanded configuration. The anchoring frame 402 is a generally tubular component or stent and forms a hollow, generally cylindrical shape around a central longitudinal axis LA. In the embodiment shown in FIGS. 22-26, the anchoring frame 402 has a bell shape or profile which includes three flared portions. More particularly, with reference to the side, top, and perspective views of FIGS. 22, 23, and 26, respectively, the anchoring frame 402 includes a first fixation portion 420A and a second fixation portion 420B which are configured to securely fix the anchoring frame 402 to tissue at a native heart valve, an integration region 422 configured to integrate the anchoring frame 402 with the valve support, and a lateral portion 424 between the first fixation portion 420A and the integration region 422.

The lateral portion 424 is similar to the lateral portion 124, and extends at an angle Θ1 relative to the central longitudinal axis LA of the anchoring frame 402. In an embodiment, angle Θ₁ is between 40° and 60° relative to the central longitudinal axis LA. In the direction from the inflow end 412 to the outflow end 410, the lateral portion 424 flares or extends radially outward at the angle Θ₁. The lateral portion 424 is a portion of the anchoring frame 402 that is generally conically shaped and extends radially outwardly in the direction of bloodflow therethrough, from the inflow end 412 to the outflow end 410. The lateral portion 424 may be generally straight, or it may be curved. In some embodiments, the lateral portion 424 is shape set with curvatures that mirror or mimic the shape of an annulus to allow for improved conformity when deployed. The lateral portion 424 may be shape set with curvatures, such as S-, U-, or hourglass shapes, to allow for increased conformity to the native heart-valve anatomy when implanted in vivo. However, this not meant to be limiting, and in other embodiments, the lateral portion 424 may be generally straight and may help the anchoring frame 402 conform to the anatomy by being relatively flexible.

As depicted, the first fixation portion 420A of the anchoring frame is integrally formed with the lateral portion 424 of the anchoring frame 302. In other embodiments, the first fixation portion 420A and the lateral portion 424 are made integral with an attachment mechanism, such as one or more hooks, hinges, or rivets. The first fixation portion 420A extends at an angle Θ2 relative to the central longitudinal axis LA of the anchoring frame 402. In an embodiment, angle Θ2 is between 10° and 30° relative to the central longitudinal axis LA. In the direction of bloodflow therethrough, from the inflow end 412 to the outflow end 410, the first fixation portion 420A flares or extends radially outward at the angle Θ2. The first fixation portion 420A may be generally straight, or it may be generally curved with one or more arcs, including S-, U-, or hourglass shapes. In some embodiments, the first fixation portion 420A is shape set with curvatures that mirror or mimic the shape of a native annulus to allow for improved conformity when deployed. The first fixation portion 420A may be shape set with curvatures, such as S-, U-, or hourglass shapes, to allow for increased conformity to the native heart-valve anatomy when implanted in vivo. However, this not meant to be limiting, and in other embodiments, the first fixation portion 420A may be generally straight and may help the anchoring frame 402 conform to the anatomy by being relatively flexible.

As depicted, the second fixation portion 420B of the anchoring frame is integrally formed with the first fixation portion 420A of the anchoring frame 302. In other embodiments, the first fixation portion 420A and the second fixation portion 420B are made integral with an attachment mechanism, such as one or more hooks, hinges, or rivets. The second fixation portion 420B extends at an angle Θ3 relative to the central longitudinal axis LA of the anchoring frame 402. In an embodiment, angle Θ3 is between 30° and 50° relative to the central longitudinal axis LA. In the direction of bloodflow therethrough, from the inflow end 412 to the outflow end 410, the second fixation portion 420B flares or extends radially outward at the angle Θ3. The second fixation portion 420B may be generally straight, or it may be generally curved with one or more arcs, including S-, U-, or hourglass shapes. In some embodiments, the second fixation portion 420B is shape set with curvatures that mirror or mimic the shape of an annulus to allow for improved conformity when deployed. The second fixation portion 420B may be shape set with curvatures, such as S-, U-, or hourglass shapes, to allow for increased conformity to the native heart-valve anatomy when implanted in vivo. However, this not meant to be limiting, and in other embodiments, the second fixation portion 420B may be generally straight and may help the anchoring frame 402 conform to the anatomy by being relatively flexible. Collectively, the first and second fixation portions 420A, 420B of the anchoring frame 402 may have a length L₁ (see FIG. 24), extending from the outflow end 410 to the junction between the first fixation portion 420A and the lateral portion 424, of about 9 mm-13 mm, for example. The second fixation portion 420B of the anchoring frame 402 may have a length L₂ (see FIG. 24), extending from the outflow end 310 to the junction between the first fixation portion 420A and the second fixation portion 420B, of about 2 mm-4 mm, for example.

The lateral portion 424, the first fixation portion 420A, and the second fixation portion 420B may thus be flared at various angles and collectively provide the anchoring frame 402 with a bell shape or profile. The bell shape or profile is configured to conform to the native annulus of, for example, a native mitral or tricuspid valve. In addition, with the fixation portions 420A, 420B flared radially outward rather than parallel to the central longitudinal axis LA, some embodiments reduce the amount of the anchoring frame 402 that protrudes or extends into or around the LVOT. Thus, obstruction of blood flow through the LVOT may be minimized or prevented with such designs.

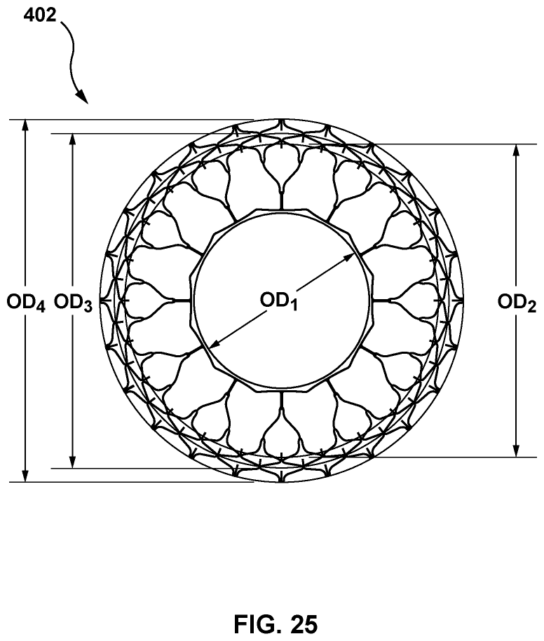

With reference to the side and top views of FIGS. 24 and 25, respectively, the bell shape or profile of the anchoring frame 402 is also apparent via a comparison of the outer diameters along the length L of the anchoring frame 402. More particularly, the anchoring frame 402 has an outer diameter OD₁ at the inflow end 412, an outer diameter OD₂ at the junction between the lateral portion 424 and the first fixation portion 420A, an outer diameter OD₃ at the junction between the first fixation portion 420A and the second fixation portion 420B, and an outer diameter OD₄ at the outflow end 410. The outer diameters increase in the direction of the bloodflow therethrough, with the outer diameter OD₂ being greater than the outer diameter OD₁ and the outer diameter OD₃ being greater than the outer diameter OD₂ and the outer diameter OD₄ being greater than the outer diameter OD₃. In an embodiment, the outer diameter OD₁ ranges between 26 mm and 30 mm, the outer diameter OD₂ ranges between 47 mm and 53 mm, the outer diameter OD₃ ranges between 50 mm and 56 mm, and the outer diameter OD₄ ranges between 55 mm and 60 mm.

The anchoring frame 402 includes openings or cells 416 that may be diamond-shaped. The anchoring frame 402 may be formed by a laser-cut manufacturing method and/or another conventional frame forming methods. For example, the anchoring frame 402 may be laser cut from a single metal tube into the desired geometry, creating a tubular scaffold of interconnected struts that form the openings 416. The anchoring frame 402 may then be shaped into a desired configuration, e.g., bell shape or profile which includes three or more tapers or flared portions at varying angles, using known shape-setting techniques for such materials.

In embodiments hereof, the anchoring frame 402 is self-expanding to return to a radially expanded state from a radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. Thus, the anchoring frame 402 has a radially compressed configuration for delivery within a delivery system and the radially expanded configuration for deployment within an annulus of the native heart valve site.

The anchoring frame 402 further includes tissue engaging elements 414, which may be the same as or similar to the tissue engaging elements 314. The tissue engaging elements 414 may be cleats or barbs disposed on an outer wall or surface of the anchoring frame 402 and extending in an upward (towards the inflow end 412) and/or radially outward direction to engage, and in some embodiments, penetrate the native tissue to facilitate retention or maintain position of the transcatheter heart valve prosthesis in a desired implanted location. In an embodiment, each tissue engaging element 414 has a blunt atraumatic unattached or free end 415.

Each tissue engaging element 414 is disposed at a node of the anchoring frame 402 or at an endmost outflow crown of the anchoring frame 402, with nodes and endmost outflow crowns being defined the same as described above with respect to nodes 380 and endmost outflow crowns 382A. Further, as best shown on FIG. 24, the fixation portions 420A, 420B of the anchoring frame 402 collectively include three rows R1, R2, R3 of tissue engaging elements 414. Row R1 of the tissue engaging elements 414 is disposed at the outflow end 410 of the anchoring frame 402. Row R2 of the tissue engaging elements 414 is disposed proximal of row R1, and row R3 of the tissue engaging elements 414 is disposed proximal of row R2. The placement of tissue engaging elements 414 within rows R1, R2, R3 may be the same as the placement of tissue engaging elements 314 within rows R1, R2, R3 described above with respect to FIGS. 19-21.

Although not shown, in some embodiments, the anchoring frame 402 further includes a brim such as brim 108 or brim 208 described above with respect to the anchoring frames 102 and 202, respectively. The brim may be coupled to and extend radially outwardly from the anchoring frame 402. In other embodiments, the anchoring frame 402 does not include a brim.

The anchoring frame 402 may also include one or more sealing members, membranes, or skirts coupled to the inner and/or outer surfaces of the anchoring frame 402. In an embodiment, the anchoring frame 402 includes a first skirt (similar to the first skirt 160) coupled to an inner surface of the anchoring frame 402. The first skirt is configured to prevent paravalvular leaks between the anchoring frame 402 and the native tissue and/or between the anchoring frame 402 and the valve support. The first skirt may be formed from a low-porosity woven fabric, such as polyester, polyethylene terephthalate (PET), or polytetrafluoroethylene (PTFE). The first skirt may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

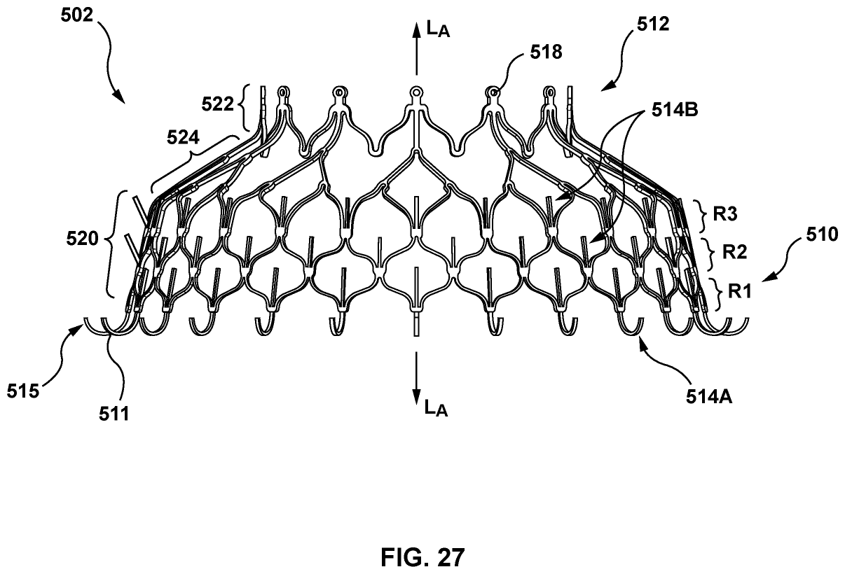

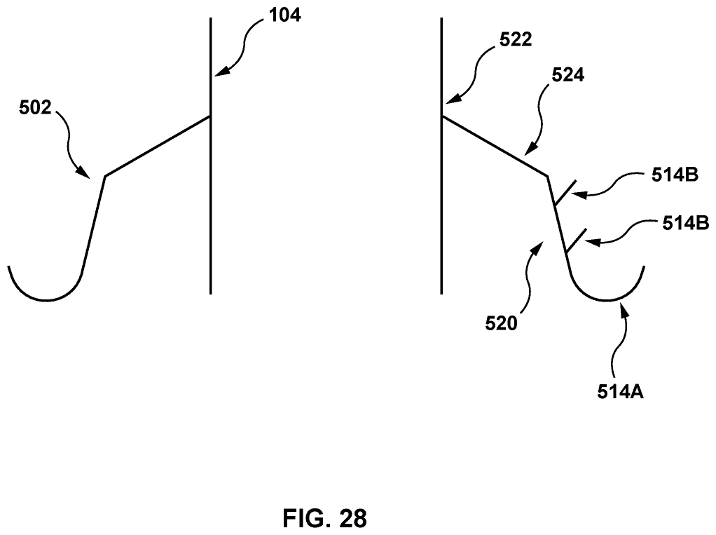

FIGS. 27 and 28 show an embodiment of an anchoring member or frame 502 for use in the transcatheter heart valve prosthesis 100. The anchoring member or frame 502 is configured to at least partially surround and be coupled to the valve support 104, as shown in FIG. 28. The anchoring frame 502 is configured for placement within a native atrioventricular valve and includes a downstream or distal or outflow end 510, and an upstream or proximal end, referred to herein as an inflow end 512. As can be seen, FIG. 27 shows the anchoring frame 502 with the inflow end 512 at the top of the figure and the outflow end 510 at the bottom of the figure. The anchoring frame 502 may have a length extending from the inflow end 512 to the outflow end 510, of about 20 mm to 25 mm, for example. The anchoring frame 502 may include a plurality of eyelets 518 disposed at the proximal/inflow end of the anchoring frame 502, for example.

The anchoring frame 502 may be the same as the anchoring frame 302 described above, except for, for example, the configuration of the tissue engaging elements along row R1 of the frame. More particularly, FIGS. 27 and 28 depict the anchoring frame 502 in its radially expanded configuration. The anchoring frame 502 is a generally tubular component or stent and forms a hollow, generally cylindrical shape around a central longitudinal axis LA. The anchoring frame 502 has a bell shape or profile which includes two flared portions. More particularly, the anchoring frame 502 includes a fixation portion 520 which may be the same as the fixation portion 320, an integration region 522 which may be the same as integration region 322, and a lateral portion 524 which may be the same as the lateral portion 324. Since these portions of the anchoring frame 502 may be the same as described with respect to the corresponding portions of the anchoring frame 302 described above, the details are not repeated here.

The depicted anchoring frame 502 includes two types of tissue engaging elements, namely tissue engaging elements 514A and tissue engaging elements 514B. More particularly, the fixation portion 520 of the anchoring frame 502 includes three rows R1, R2, R3 of tissue engaging elements with row R1 terminating at the tissue engaging elements 514A and rows R2, R3 including the tissue engaging elements 514B. Row R1 of the tissue engaging elements 514A is disposed at the outflow end 510 of the anchoring frame 502. Row R2 of the tissue engaging elements 514B is disposed proximal of row R1, and row R3 of the tissue engaging elements 514B is disposed proximal of row R2. Each tissue engaging element 514B is disposed at a node of the anchoring frame 502 and each tissue engaging elements 514A is disposed at an endmost outflow crown of the anchoring frame 502, with nodes and endmost outflow crowns being defined the same as described above with respect to nodes 380 and endmost outflow crowns 382A. In another embodiment hereof (not shown), the anchoring frame 502 may include both tissue engaging elements 514A and tissue engaging elements 514B at the endmost outflow crowns. Stated another way, row R1 may include both tissue engaging elements 514A and tissue engaging elements 514B at the endmost outflow crowns rather than just tissue engaging elements 514A.

The tissue engaging elements 514B of rows R2, R3 may be the same as the tissue engaging elements 314 of the anchoring frame 302. Thus, the tissue engaging elements 514B may be cleats or barbs disposed on an outer wall or surface of the anchoring frame 502 and extending in an upward (towards the inflow end 512) and/or radially outward direction to engage, and in some embodiments, penetrate the native tissue to facilitate retention or maintain position of the transcatheter heart valve prosthesis in a desired implanted location. Since tissue engaging elements 514B of the anchoring frame 502 may be the same as the corresponding tissue engaging elements 314 of the anchoring frame 302 described above, the details are not repeated here.

The tissue engaging elements 514A of row R1 are depicted as hook-shaped cleats extending from the outflow end 510 of the anchoring frame 502. More particularly, as shown, each tissue engaging element 514A distally extends from an endmost outflow crown of the anchoring frame 502 in a downstream direction, includes a curved portion 511 at a distal-most portion thereof which extends radially outward relative to the endmost outflow crown of the anchoring frame 502, and terminates at a free or unattached end 515. The curved portion 511 extends in an upstream direction, such that the free ends 515 of the tissue engaging elements 514A are disposed proximal to the curved portion 511. In an embodiment, the free ends 515 are disposed at the same or similar longitudinal or axial depth as the endmost outflow crowns of the anchoring frame 502. In an embodiment, each tissue engaging element 514A along the row R1 has the same or similar curvature and configuration. Collectively, the tissue engaging elements 514A may form an atraumatic flange at the outflow end 510 that is configured to better conform to the native annulus which may be configured to improve sealing and migration resistance of the prosthesis.

In a radially compressed state or delivery configuration (not shown), the tissue engaging elements 514A may be generally straightened and may distally extend from a distal end of the anchoring frame 502. In such configurations, the tissue engaging elements 514A are self-expanding to return to a radially expanded state (shown in FIGS. 27 and 28) from the radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. Thus, the tissue engaging elements 514A have a radially compressed configuration for delivery within a delivery system and the radially expanded configuration for deployment within an annulus of the native heart valve site.

Although not shown, in some embodiments, the anchoring frame 502 further includes a brim such as brim 108 or brim 208 described above with respect to the anchoring frames 102 and 202, respectively. The brim may be coupled to and extend radially outwardly from the anchoring frame 502. In other embodiments, the anchoring frame 502 does not include a brim.

The anchoring frame 502 may also include one or more sealing members, membranes, or skirts coupled to the inner and/or outer surfaces of the anchoring frame 502. In an embodiment, the anchoring frame 502 includes a first skirt (similar to the first skirt 160) coupled to an inner surface of the anchoring frame 502. The first skirt is configured to prevent paravalvular leaks between the anchoring frame 502 and the native tissue and/or between the anchoring frame 502 and the valve support. The first skirt may be formed from a low-porosity woven fabric, such as polyester, polyethylene terephthalate (PET), or polytetrafluoroethylene (PTFE), and may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

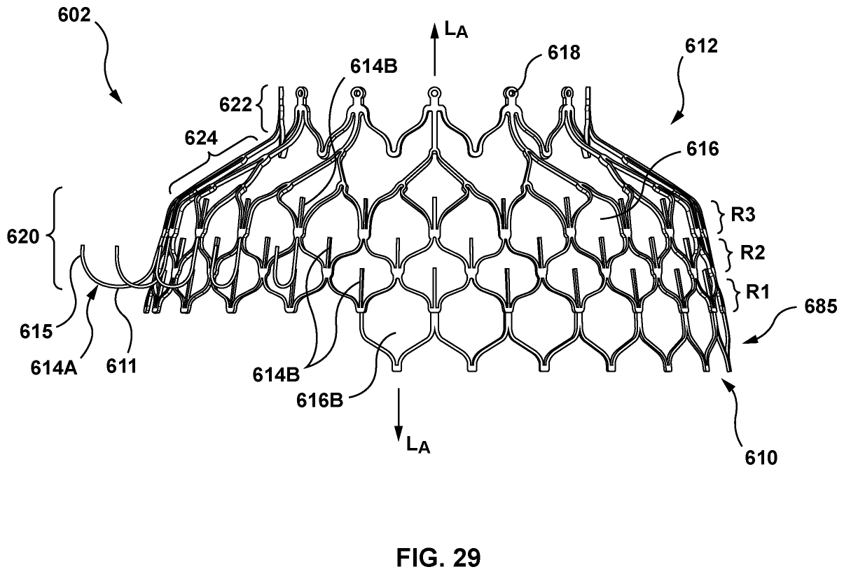

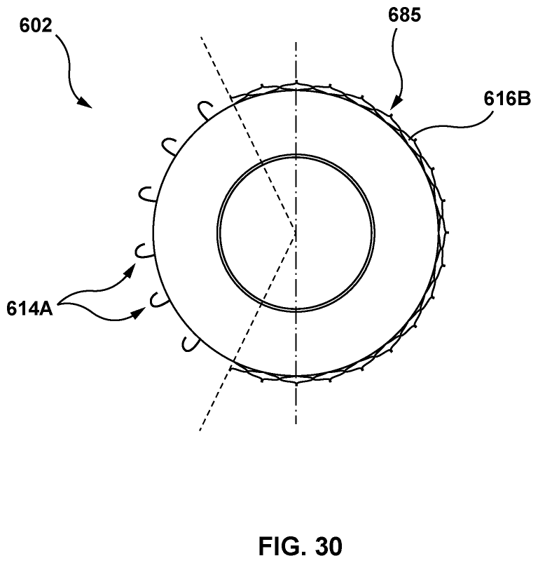

FIGS. 29 and 30 show an embodiment of an anchoring member or frame 602 for use in the transcatheter heart valve prosthesis 100. The anchoring member or frame 602 is configured to at least partially surround and be coupled to the valve support 104. FIGS. 29 and 30 show the anchoring frame 602 without the valve support attached, for clarity. The anchoring frame 602 is configured for placement within a native atrioventricular valve anatomy and includes a downstream or distal or outflow end 610, and an upstream or proximal end, for example an inflow end 612. As can be seen, FIG. 29 shows the anchoring frame 602 with the inflow end 612 at the top of the figure and the outflow end 610 at the bottom of the figure. The anchoring frame 602 may have a length extending from the inflow end 612 to the outflow end 610, of about 23 mm-28 mm, for example. The anchoring frame 602 may include a plurality of eyelets 618 disposed at the proximal/inflow end of the anchoring frame 602, for example.

The anchoring frame 602 may be the same as the anchoring frame 302 described above, except for certain differences described herein, for example. More particularly, FIGS. 29 and 30 depict the anchoring frame 602 in its radially expanded configuration. The anchoring frame 602 is a generally tubular component or stent and forms a hollow, generally cylindrical shape around a central longitudinal axis LA. The anchoring frame 602 includes openings or cells 616 that may be diamond-shaped and similar to the cells 316 described above. The anchoring frame 602 has a bell shape or profile which includes two flared portions. More particularly, the anchoring frame 602 includes a fixation portion 620 which may be the same as the fixation portion 320, an integration region 622 which may be the same as integration region 322, and a lateral portion 624 which may be the same as the lateral portion 324. Since these portions of the anchoring frame 602 may be the same as the corresponding portions of the anchoring frame 302 described above, the details hereof are not repeated.

To improve sealing and migration resistance of the transcatheter prosthesis, for example, the anchoring frame 602 may include a distal extension 685 of the fixation portion 620 at the outflow end 610. The distal extension 685 includes an additional row of cells or openings 616B that are formed integrally with all or a portion of the fixation portion 620 of the anchoring frame 602. The additional row of cells 616B may or may not extend around the entire circumference of the outflow end 610 of the anchoring frame 602, and it does not extend around the entire circumference of the outflow end 610 in the embodiment depicted here. More particularly, as best shown on the top view of FIG. 30, the additional row of cells 616B extends around between 190° and 225° of the circumference of the outflow end 610 of the anchoring frame 602. In an embodiment, the additional row of cells 616B is configured to be positioned adjacent to the posterior leaflet of a native mitral valve and therefore the anchoring frame 602 includes additional surface area for posterior wall fixation or sealing. As a result, the additional row of cells 616B is configured to improve sealing and migration resistance of the anchoring frame 602.

In addition, the anchoring frame 602 shown here includes two types of tissue engaging elements, namely tissue engaging elements 614A and tissue engaging elements 614B. More particularly, the fixation portion 620 of the anchoring frame 602 includes three rows R1, R2, R3 of tissue engaging elements with rows R1, R2 including the tissue engaging elements 614B and row R3 including both tissue engaging elements 614A and tissue engaging elements 614B. In this embodiment, the additional row of cells 616B of the distal extension 685 does not include any additional tissue engaging elements. Row R1 of the tissue engaging elements 614A is disposed near the outflow end 610 of the anchoring frame 602. Row R2 of the tissue engaging elements 614B is disposed proximal of row R1, and row R3 of the tissue engaging elements 614B is disposed proximal of row R2. Each tissue engaging element 614B is disposed at a node or an endmost outflow crown of the anchoring frame 602 and each tissue engaging elements 614A is disposed at a node of the anchoring frame 602, with nodes and endmost outflow crowns being defined the same as described above with respect to nodes 380 and endmost outflow crowns 382A. In other embodiments, tissue engaging elements are disposed at various, but not all, nodes of the anchoring frame. In other embodiments, tissue engaging elements are disposed on struts between nodes or between a distalmost node and the endmost outflow crown of the anchoring frame. With respect to the row R3, each node along the row R3 includes a tissue engaging element 614B while only a portion of the nodes along the row R3 includes the tissue engaging elements 614A. The nodes along the row R3 which include the tissue engaging elements 614A thus include both types of tissue engaging elements 614A, 614B. Particularly, when both types of tissue engaging elements 614A, 614B are present on the same node, the tissue engaging nodes 614B generally extend from a proximal end of the node and the tissue engaging nodes 614A generally extend from a distal end of the node.

The tissue engaging elements 614B of rows R1, R2, R3 may be the same as the tissue engaging elements 314 of the anchoring frame 302. Thus, the tissue engaging elements 614B may be cleats or barbs disposed on an outer wall or surface of the anchoring frame 602 and extending in an upward (towards the inflow end 612) and/or radially outward direction to engage, and in some embodiments, penetrate the native tissue to facilitate retention or maintain position of the prosthesis in a desired implanted location. Since tissue engaging elements 614B of the anchoring frame 602 may be the same as the corresponding tissue engaging elements 314 of the anchoring frame 302 described above, the details are not repeated here.

The tissue engaging elements 614A of row R3 are hook-shaped cleats extending from nodes of the anchoring frame 602. More particularly, each tissue engaging element 614A distally extends from a node of the anchoring frame 602 in a downstream direction, includes a curved portion 611 at a distal-most portion thereof which extends radially outward relative to the anchoring frame 602, and terminates at a free or unattached end 615. The curved portion 611 curves into an upstream direction, such that the free ends 615 of the tissue engaging elements 614A are disposed proximal to the curved portion 611. In an embodiment, the free end 615 of each tissue-engaging element 614A is disposed at the same or similar longitudinal or axial depth as the node from which the tissue engaging element 614A extends. In other embodiments, the free end 615 may be disposed proximal or distal to the node from which the tissue engaging element 614A extends. In an embodiment, each tissue engaging element 614A along the row R3 has the same curvature and configuration. In other embodiments, tissue engaging elements 614A have varying curvatures and configurations.

As best shown in the top view of FIG. 30, the tissue engaging elements 614A do not extend around the entire circumference of the outflow end 610 of the anchoring frame 602 in the embodiment depicted here. More particularly, the tissue engaging elements 614A extend around between 135° and 170° of the circumference of the anchoring frame 602. In an embodiment, the tissue engaging elements 614A are configured to be positioned adjacent to the anterior leaflet of a native mitral valve and form an atraumatic anterior flange that is configured to conform to the native annulus. As a result, the tissue engaging elements 614A is configured to improve sealing and migration resistance of the anchoring frame 602.

In an embodiment, as best shown on FIG. 30, the tissue engaging elements 614A and the additional row of cells 616B do not circumferentially overlap and collectively extend over an entire circumference of the anchoring frame 602. For example, in an embodiment, the additional row of cells 616B may extend over 200° of the circumference of the anchoring frame 602 while the tissue engaging elements 614A extend over 160° of the circumference of the anchoring frame 602, thereby collectively covering a full 360° of the circumference of the anchoring frame 602. In an axial or longitudinal direction, the tissue engaging elements 614A are disposed proximal to the additional row of cells 616B.

In a radially compressed state or delivery configuration (not shown), the tissue engaging elements 614A may be generally straightened and may distally extend from the respective node of the anchoring frame 602. The tissue engaging elements 614A are self-expanding to return to a radially expanded state (shown in FIGS. 29 and 30) from the radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. Thus, the tissue engaging elements 614A have a radially compressed configuration for delivery within a delivery system and the radially expanded configuration for deployment within an annulus of the native heart valve site.

Although not shown, in some embodiments, the anchoring frame 602 further includes a brim such as brim 108 or brim 208 described above with respect to the anchoring frames 102 and 202, respectively. The brim may be coupled to and extend radially outwardly from the anchoring frame 602. In other embodiments, the anchoring frame 602 does not include a brim.

The anchoring frame 602 may also include one or more sealing members, membranes, or skirts coupled to the inner and/or outer surfaces of the anchoring frame 602. In an embodiment, the anchoring frame 602 includes a first skirt (similar to the first skirt 160) coupled to an inner surface of the anchoring frame 602. The first skirt is configured to prevent paravalvular leaks between the anchoring frame 602 and the native tissue and/or between the anchoring frame 602 and the valve support. The first skirt may be formed from a low-porosity woven fabric, such as polyester, polyethylene terephthalate (PET), or polytetrafluoroethylene (PTFE), which may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

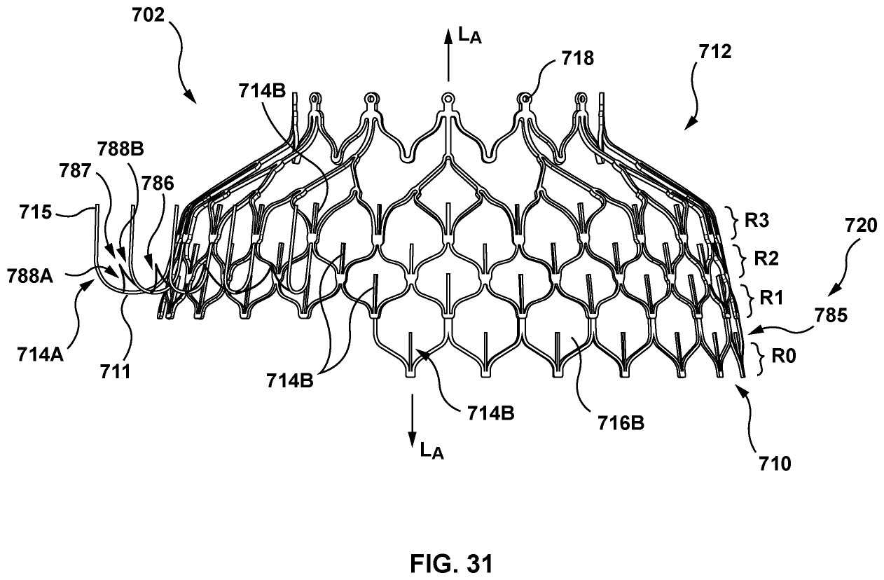

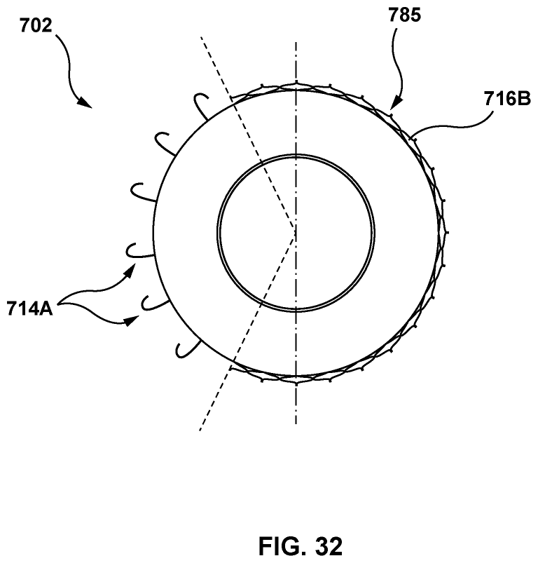

FIGS. 31 and 32 show an embodiment of an anchoring member or frame 702 for use in the prosthesis 100. The anchoring member or frame 702 is configured to at least partially surround and be coupled to the valve support 104. FIGS. 31 and 32 show the anchoring frame 702 without the valve support attached, for clarity. The anchoring frame 702 is configured for placement within a native atrioventricular valve and includes a downstream or distal or outflow end 710, and an upstream or proximal or inflow end 712. As shown in FIG. 31, the anchoring frame 702 with the inflow end 712 is at the top of the figure and the outflow end 710 at the bottom of the figure of this embodiment. The anchoring frame 702 may have a length extending from the inflow end 712 to the outflow end 710, of about 23 mm-28 mm, for example. The anchoring frame 702 may include a plurality of eyelets 718 disposed at the proximal/inflow end of the anchoring frame 702, for example.

The anchoring frame 702 may be the same as the anchoring frame 602 described above, except for, for example, the differences described herein. More particularly, similar to the anchoring frame 602, the anchoring frame 702 includes a distal extension 785 of a fixation portion 720 at the outflow end 710. The distal extension 785 includes an additional row of cells or openings 716B that are formed integrally with the fixation portion 720 of the anchoring frame 702. The additional row of cells 716B does not extend around the entire circumference of the outflow end 710 of the anchoring frame 702 in this embodiment. More particularly, as best shown in the top view of FIG. 32, the additional row of cells 716B extends around between 190° and 225° of the circumference of the outflow end 710 of the anchoring frame 702. In an embodiment, the additional row of cells 716B is configured to positioned adjacent to the posterior leaflet of a native mitral valve and therefore the anchoring frame 702 includes additional surface area for posterior wall fixation or sealing. As a result, the additional row of cells 716B is configured to improve sealing and migration resistance of the anchoring frame 702.

In addition, similar to the anchoring frame 602, the anchoring frame 702 also includes two types of tissue engaging elements, namely tissue engaging elements 714A and tissue engaging elements 714B. In this embodiment, the anchoring frame 702 includes four rows R0, R1, R2, R3 of tissue engaging elements with rows R0, R1, R2 including the tissue engaging elements 714B and row R3 including both tissue engaging elements 714A and tissue engaging elements 714B. In this embodiment, the additional row of cells 716B of the distal extension 785 includes the row R0 of tissue engaging elements 714B. Row R1 is disposed proximal of row R0, row R2 is disposed proximal of row R1, and row R3 is disposed proximal of row R2. Each tissue engaging element 714B is disposed at a node or an endmost outflow crown of the anchoring frame 702 and each tissue engaging element 714A is disposed at a node of the anchoring frame 702, with nodes and endmost outflow crowns being defined the same as described above with respect to nodes 380 and endmost outflow crowns 382A. In other embodiments, tissue engaging elements are disposed at various, but not all, nodes of the anchoring frame. In other embodiments, tissue engaging elements are disposed on struts between nodes or between a distalmost node and the endmost outflow crown of the anchoring frame. With respect to the row R3, each node along the row R3 includes a tissue engaging element 714B while only a portion of the nodes along the row R3 includes the tissue engaging elements 714A. The nodes along the row R3 which include the tissue engaging elements 714A thus include both types of tissue engaging elements 714A, 714B. Particularly, when both types of tissue engaging elements 714A, 714B are present on the same node, the tissue engaging nodes 714B generally extend from a proximal end of the node and the tissue engaging nodes 714A generally extend from a distal end of the node.

The tissue engaging elements 714B of rows R0, R1, R2, R3 may be the same as the tissue engaging elements 314 of the anchoring frame 302. Thus, the tissue engaging elements 714B may be cleats or barbs disposed on an outer wall or surface of the anchoring frame 702 and extending in an upward (towards the inflow end 712) and/or radially outward direction to engage, and in some embodiments, penetrate the native tissue to facilitate retention or maintain position of the prosthesis in a desired implanted location. Since tissue engaging elements 714B of the anchoring frame 702 may be the same as the corresponding tissue engaging elements 314 of the anchoring frame 302 described above, the details are not repeated here.

The tissue engaging elements 714A of row R3 are hook-shaped cleats extending from nodes of the anchoring frame 702. More particularly, each tissue engaging element 714A distally extends from a node of the anchoring frame 702 in a downstream direction, includes a curved portion 711 at a distal-most portion thereof which extends radially outward relative to the anchoring frame 702, and terminates at a free or unattached end 715. The curved portion 711 curves into an upstream direction, such that the free ends 715 of the tissue engaging elements 714A are disposed proximal to the curved body 711. As shown on FIG. 31, in this embodiment, the free end 715 is disposed at further upstream than the free end 615 of the tissue engaging elements 614A. In this embodiment, the free end 715 is disposed proximal to the node from which the tissue engaging element 714A extends and may be disposed at the same or similar longitudinal or axial depth as the free end of the tissue engaging element 714B extending from the same node as the tissue engaging element 714A. In other embodiments, the free end 715 may be disposed proximal or distal to the node from which the tissue engaging element 714A extends. In an embodiment, each tissue engaging element 714A along the row R3 has the same curvature and configuration. In other embodiments, tissue engaging elements 714A have varying curvatures and configurations.

In this embodiment, V-shaped connectors or supports 786 circumferentially extend between pairs of adjacent tissue engaging element 714A. Each V-shaped connector 786 includes an apex 787 and a pair of struts or legs 788A, 788B that distally extend from the apex 787. Each leg 788A, 788B is attached to or integrally formed with a distal-most portion of the curved body 711 of a tissue engaging element 714A. In an embodiment, the legs 788A, 788B may be substantially straight, or may be curved as depicted in FIG. 31. The legs 788A, 788B may be shape set with curvatures to allow for increased flexibility when implanted in vivo. The apex 787 of the V-shaped connector 786 forms a proximal-most end of the V-shaped connector 786 in this embodiment. In an embodiment, the free ends 715 of the tissue engaging elements 714A are disposed proximal to the apexes 787 of the V-shaped connectors 786.

As shown in FIGS. 31 and 32, the tissue engaging elements 714A do not extend around the entire circumference of the outflow end 710 of the anchoring frame 702 in the embodiment depicted here. More particularly, the tissue engaging elements 714A extend around between 135° and 170° of the circumference of the anchoring frame 702. In an embodiment, the tissue engaging elements 714A are configured to positioned adjacent to the anterior leaflet of a native mitral valve and form an atraumatic anterior flange that is configured to better conform to the native mitral annulus. As a result, the tissue engaging elements 714A are configured to improve sealing and migration resistance of the anchoring frame 702.

In an embodiment, as best shown on FIG. 32, the tissue engaging elements 714A and the additional row of cells 716B do not circumferentially overlap and collectively extend over an entire circumference of the anchoring frame 702. For example, in an embodiment, the additional row of cells 716B may extend along 200° of the circumference of the anchoring frame 702 while the tissue engaging elements 714A extend along 160° of the circumference of the anchoring frame 702, thereby collectively covering a full 360° of the anchoring frame. In an axial or longitudinal direction, the tissue engaging elements 714A are disposed proximal to the additional row of cells 716B.

In a radially compressed state or delivery configuration (not shown), the tissue engaging elements 714A may be generally straightened and may distally extend from the respective node of the anchoring frame 702. The tissue engaging elements 714A as well as the V-shaped connectors 786 are self-expanding to return to a radially expanded state (shown in FIGS. 31 and 32) from the radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. Thus, the tissue engaging elements 714A have a radially compressed configuration for delivery within a delivery system and the radially expanded configuration for deployment within an annulus of the native heart valve site.

Although not shown, in some embodiments, the anchoring frame 702 further includes a brim such as brim 108 or brim 208 described above with respect to the anchoring frames 102 and 202, respectively. The brim may be coupled to and extend radially outwardly from the anchoring frame 702. In other embodiments, the anchoring frame 702 does not include a brim.

The anchoring frame 702 may also include one or more sealing members, membranes, or skirts coupled to the inner and/or outer surfaces of the anchoring frame 702. In an embodiment, the anchoring frame 702 includes a first skirt (similar to the first skirt 160) coupled to an inner surface of the anchoring frame 702. The first skirt is configured to prevent paravalvular leaks between the anchoring frame 702 and the native tissue and/or between the anchoring frame 702 and the valve support. The first skirt may be formed from a low-porosity woven fabric, such as polyester, polyethylene terephthalate (PET), or polytetrafluoroethylene (PTFE), which may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

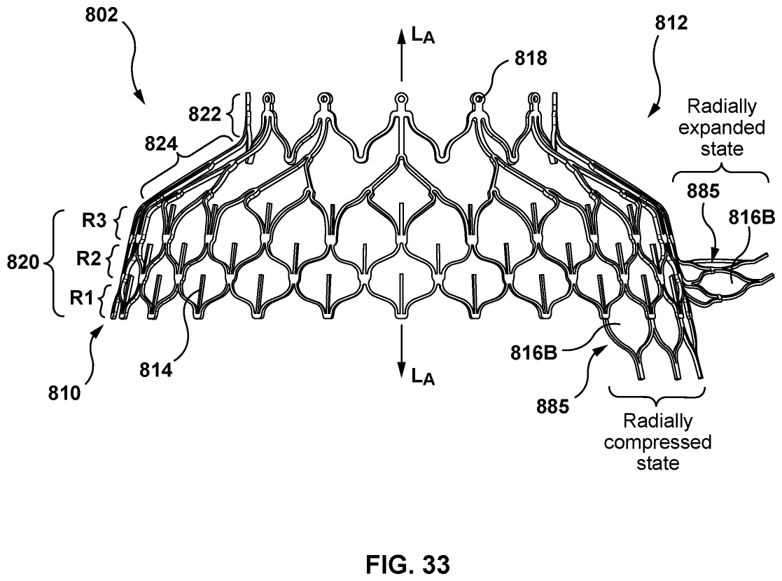

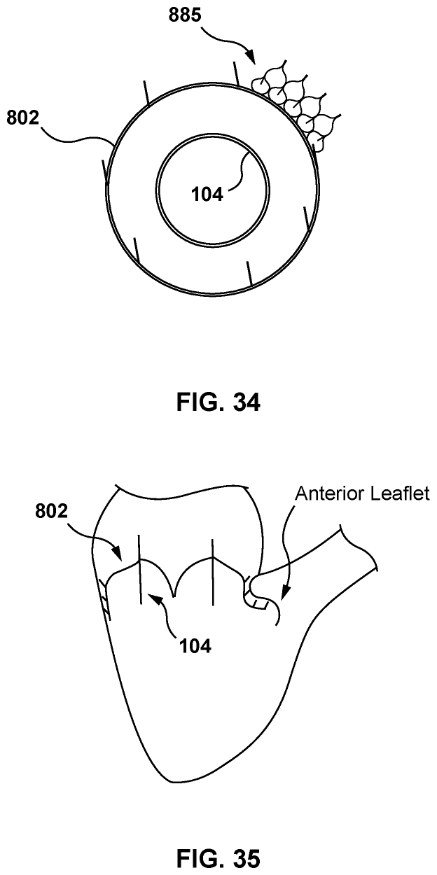

FIGS. 33-35 show an embodiment of an anchoring member or frame 802 for use in the prosthesis 100. The anchoring member or frame 802 is configured to at least partially surround and be coupled to the valve support 104, as shown in FIGS. 34 and 35. The anchoring frame 802 is configured for placement within a native atrioventricular valve and includes a downstream or distal or outflow end 810, and an upstream or proximal end, referred to herein as an inflow end 812. As can be seen, FIG. 33 shows the anchoring frame 802 with the inflow end 812 at the top of the figure and the outflow end 810 at the bottom of the figure. The anchoring frame 802 may have a length extending from the inflow end 812 to the outflow end 810, of about 20 mm-25 mm, for example. The anchoring frame 802 may include a plurality of eyelets 818 disposed at the proximal/inflow end of the anchoring frame 802, for example.

The anchoring frame 802 may be the same as the anchoring frame 302 described above, except for, for example, the differences described herein. More particularly, FIGS. 33-35 depict the anchoring frame 802 in its radially expanded configuration. The anchoring frame 802 is a generally tubular component or stent and forms a hollow cylindrical shape around a central longitudinal axis LA. In some embodiments, the anchoring frame 802 has a hollow cylindrical shape that resembles a D-shape, similar to the shape of a native mitral valve annulus, for example. The anchoring frame 802 has a bell shape or profile which includes two flared portions. More particularly, the anchoring frame 802 includes a fixation portion 820 which is the same as the fixation portion 320, an integration region 822 which is the same as integration region 322, and a lateral portion 824 which may be the same as the lateral portion 324. Since these portions of the anchoring frame 802 may be the same as the corresponding portions of the anchoring frame 302 described above, the details are not repeated here.

To improve sealing and migration resistance of the prosthesis, for example, the anchoring frame 802 may include a distal extension 885 of the fixation portion 820 at the outflow end 810. The distal extension 885 includes an additional row of cells or openings 816B that are formed integrally with all or a portion of the fixation portion 820 of the anchoring frame 802. The additional row of cells 816B may or may not extend around the entire circumference of the outflow end 810 of the anchoring frame 802, and it does not extend around the entire circumference of the outflow end 610 in the embodiment depicted here. More particularly, as best shown on the top view of FIG. 34, the additional row of cells 816B extends around between 30° and 60° of the circumference of the outflow end 810 of the anchoring frame 802.

The distal extension 885 has a radially compressed state or delivery configuration in which it is generally straightened and distally extends from the fixation portion 820 of the anchoring frame 802. The distal extension 885 is shape set in a deployed or radially expanded state, and is self-expanding to return to the radially expanded state from the radially compressed state. In an embodiment, the distal extension 885 in the radially expanded state extends generally perpendicular to the longitudinal axis LA of the anchoring frame 802. The distal extension 885 may be made from materials such as, but not limited to stainless steel, a pseudo-elastic metal such as a nickel titanium alloy (e.g., NITINOL), or a so-called super alloy, which may have a base metal of nickel, cobalt, chromium, or other metal. Thus, the distal extension 885 has a radially compressed configuration for delivery within a delivery system and the radially expanded configuration for deployment within an annulus of the native heart valve site.

FIG. 33 depicts the transition of the distal extension 885 from the radially compressed state to the radially expanded state. More particularly, when released from a delivery system, the distal extension 885 bends radially outward such that the distal extension 885 moves toward the radially expanded state thereof. With reference to FIG. 35, the distal extension 885 is configured to be positioned adjacent to the anterior leaflet of a native mitral valve. As a result, when deployed in vivo, the distal extension 885 is configured to contact the anterior leaflet of a native mitral valve and may or may not reach the shape set radially expanded state in which the distal extension 885 extends generally perpendicular to the longitudinal axis LA of the anchoring frame 802. Once deployed, the distal extension 885 functions to move or push the anterior leaflet away from the anchoring frame 802 and thus prevent the anterior leaflet from blocking or obstructing the LVOT. In addition, since the distal extension 885 is configured to engage the anterior leaflet upon deployment, the distal extension is configured to improve sealing and migration resistance of the anchoring frame 802.

The anchoring frame 802 further includes tissue engaging elements 814, which may be the same as or similar to the tissue engaging elements 314. For example, tissue engaging elements 814 may be cleats or barbs disposed on an outer wall or surface of the anchoring frame 802 and extending in an upward (towards the inflow end 812) and/or radially outward direction to engage, and in some embodiments, penetrate the native tissue to facilitate retention or maintain position of the transcatheter heart valve prosthesis in a desired implanted location. Each tissue engaging element 814 is disposed at a node of the anchoring frame 802 or at an endmost outflow crown of the anchoring frame 802, with nodes and endmost outflow crowns being as described above with respect to nodes 380 and endmost outflow crowns 382A. Row R1 of the tissue engaging elements 814 is disposed at the outflow end 810 of the anchoring frame 802. Row R2 of the tissue engaging elements 814 is disposed proximal of row R1, and row R3 of the tissue engaging elements 814 is disposed proximal of row R2. The placement of tissue engaging elements 814 within rows R1, R2, R3 may be the same as the placement of tissue engaging elements 314 within rows R1, R2, R3 described above with respect to FIGS. 19-21. Since tissue engaging elements 814 of the anchoring frame 802 may be the same as the corresponding tissue engaging elements 314 of the anchoring frame 302 described above, the details are not repeated here.

Although not shown, in some embodiments, the anchoring frame 802 further includes a brim such as brim 108 or brim 208 described above with respect to the anchoring frames 102 and 202, respectively. The brim may be coupled to and extend radially outwardly from the anchoring frame 802. In other embodiments, the anchoring frame 802 does not include a brim.

The anchoring frame 802 may also include one or more sealing members, membranes, or skirts coupled to the inner and/or outer surfaces of the anchoring frame 802. In an embodiment, the anchoring frame 802 includes a first skirt (similar to the first skirt 160) coupled to an inner surface of the anchoring frame 802. The first skirt is configured to prevent paravalvular leaks between the anchoring frame 802 and the native tissue and/or between the anchoring frame 802 and the valve support. The first skirt may be formed from a low-porosity woven fabric, such as polyester, polyethylene terephthalate (PET), or polytetrafluoroethylene (PTFE), which may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

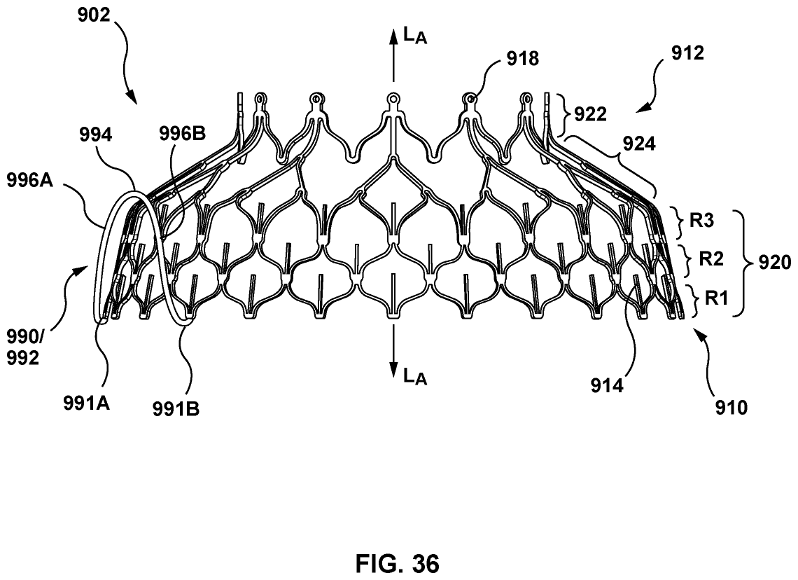

FIG. 36 shows an embodiment of an anchoring member or frame 902 for use in the prosthesis 100. The anchoring member or frame 902 is configured to at least partially surround and be coupled to the valve support 104. FIG. 36 shows the anchoring frame 702 without the valve support attached, for clarity. The anchoring frame 902 is configured for placement within a native atrioventricular valve and includes a downstream or distal or outflow end 910, and an upstream or proximal or inflow end 912. FIG. 36 shows the anchoring frame 902 with the inflow end 912 at the top of the figure and the outflow end 910 at the bottom of the figure. The anchoring frame 902 may have a length extending from the inflow end 912 to the outflow end 910, of about 20 mm-25 mm, for example. The anchoring frame 902 may include a plurality of eyelets 919 disposed at the proximal/inflow end of the anchoring frame 902, for example.

The anchoring frame 902 may be the same as the anchoring frame 302 described above, except for, for example, the differences described herein. More particularly, FIG. 36 depicts the anchoring frame 902 in its radially expanded configuration. The anchoring frame 902 is a generally tubular component or stent and forms a hollow cylindrical shape around a central longitudinal axis LA. In some embodiments, the anchoring frame 902 has a hollow cylindrical shape that resembles a D-shape, similar to the shape of a native mitral valve annulus, for example. The anchoring frame 902 has a bell shape or profile which includes two flared portions. More particularly, the anchoring frame 902 includes a fixation portion 920 which may be the same as the fixation portion 320, an integration region 922 which may be the same as integration region 322, and a lateral portion 924 which may be the same as the lateral portion 324. Since these portions of the anchoring frame 902 may be the same as the corresponding portions of the anchoring frame 302 described above, the details are not repeated here.

To improve sealing and migration resistance of the transcatheter heart valve prosthesis, the anchoring frame 902 includes at least one clip 990 at the outflow end 910. The clip 990 is attached to or formed integrally with the fixation portion 920 of the anchoring frame 902. The clip 990 may be attached or integrated using an attachment mechanism, such as one or more hooks, hinges, or rivets.

In a radially compressed state or delivery configuration (not shown), the clip 990 may be generally straightened and may distally extend from the outflow end 910 of the anchoring frame 602. The clip 990 is self-expanding to return to a radially expanded state (shown in FIG. 36) from the radially compressed state and may be made from materials such as, but not limited to stainless steel, a pseudo-elastic metal such as a nickel titanium alloy (e.g. NITINOL), or a so-called super alloy, which may have a base metal of nickel, cobalt, chromium, or other metal. When released from a delivery system, the clip 990 bends radially outward such that the clip 990 moves toward the radially expanded state thereof. In an embodiment, the clip 990 is configured to capture or extend around a leaflet of a native valve. Once deployed at the site of a native mitral valve, for example, the clip 990 functions to move or push the native leaflet away from the anchoring frame 902 and thus prevent the anterior leaflet from blocking or obstructing the LVOT. In addition, since the clip 990 is configured to capture or clip onto a native leaflet after deployment, the clip 990 is configured to improve sealing and migration resistance of the anchoring frame 902. In another embodiment, the clip 990 may engage or extend around chordae tendineae rather than, or in addition to, a leaflet of a native valve.

FIG. 36 depicts the radially expanded state of the clip 990. The clip 990 may be considered to include a pair of hinged or curved portions 991A, 991B that are attached to or integrally formed with the anchoring frame 702 at circumferentially spaced apart locations of the anchoring frame 702. Each curved portion 991A, 991B distally extends from the outflow end 910 of the anchoring frame 902 in a downstream direction, and collectively form a distal-most portion of the clip 990 when the clip 990 is in the radially expanded state. Each curved portion 991A, 991B extends radially outward relative to the anchoring frame 902, and are integrally formed with a U-shaped body 992 of the clip 990. The U-shaped body 992 proximally extends from the curved portions 991A, 991B in an upstream direction. The U-shaped body 992 includes an apex or peak 994 and a pair of struts or legs 996A, 996B extending between the peak 994 and curved portions 991A, 991B, respectively. The peak 994 forms a proximal-most end of the clip 990. In an embodiment, when the clip 990 is in the radially deployed state, the peak 994 may be disposed at the same or similar longitudinal or axial depth as the junction between the fixation portion 920 and the lateral portion 924 of the anchoring frame 902. Although FIG. 36 depicts the anchoring frame 902 with a U-shaped body 992 with a single apex or peak 994, other embodiments include frames having a body or bodies of other shapes. Shapes such as Us, Vs, Ws, sine-wave, saw-tooth, and etc. are all contemplated for clip 990. Clip 990 may also include cleats or barbs, the same as or similar to those described above.

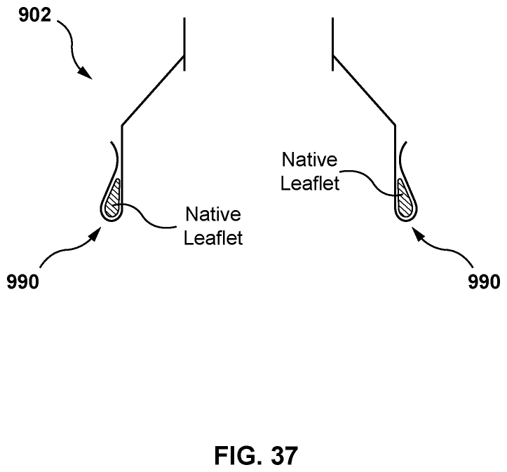

Although FIG. 36 depicts the anchoring frame 902 with a single clip 990, in another embodiment hereof as shown in the schematic side view of FIG. 37, the anchoring frame 902 includes a pair of diametrically opposing clips 990. For example, each clip 990 may be configured to capture or extend around a leaflet of a native mitral valve, i.e., the anterior and posterior leaflets of the native mitral valve. In another embodiment (not shown), the anchoring frame 990 may include a plurality of clips 990 spaced around a circumference of the anchoring frame 902 at the outflow end 910 thereof. Even where only two clips are positioned to capture or extend around a native leaflet, additional clips may still engage annular tissue and/or chordae to improve sealing and migration resistance of the anchoring frame 902.

The anchoring frame 902 further includes tissue engaging elements 914, which may be the same as or similar to the tissue engaging elements 314. The tissue engaging elements 914 may be cleats or barbs disposed on an outer wall or surface of the anchoring frame 902 and extending in an upward (towards the inflow end 912) and/or radially outward direction to engage, and in some embodiments, penetrate the native tissue to facilitate retention or maintain position of the transcatheter heart valve prosthesis in a desired implanted location. Each tissue engaging element 914 shown in FIG. 36 is disposed at a node of the anchoring frame 902 or at an endmost outflow crown of the anchoring frame 902, with nodes and endmost outflow crowns being defined the same as described above with respect to nodes 380 and endmost outflow crowns 382A. Row R1 of the tissue engaging elements 914 is disposed at the outflow end 910 of the anchoring frame 902. Row R2 of the tissue engaging elements 914 is disposed proximal of row R1, and row R3 of the tissue engaging elements 914 is disposed proximal of row R2. The placement of tissue engaging elements 914 within rows R1, R2, R3 may be the same as the placement of tissue engaging elements 314 within rows R1, R2, R3 described above with respect to FIGS. 19-21. Since tissue engaging elements 914 of the anchoring frame 902 may be the same as the corresponding tissue engaging elements 314 of the anchoring frame 302 described above, the details are not repeated here.

Although not shown, in some embodiments, the anchoring frame 902 further includes a brim such as brim 108 or brim 208 described above with respect to the anchoring frames 102 and 202, respectively. The brim may be coupled to and extend radially outwardly from the anchoring frame 902. In other embodiments, the anchoring frame 902 does not include a brim.

The anchoring frame 902 may also include one or more sealing members, membranes, or skirts coupled to the inner and/or outer surfaces of the anchoring frame 902. In an embodiment, the anchoring frame 902 includes a first skirt (similar to the first skirt 160) coupled to an inner surface of the anchoring frame 902. The first skirt is configured to prevent paravalvular leaks between the anchoring frame 902 and the native tissue and/or between the anchoring frame 902 and the valve support. The first skirt may be formed from a low-porosity woven fabric, such as polyester, polyethylene terephthalate (PET), or polytetrafluoroethylene (PTFE), which may create a one-way fluid passage when attached to the frame. In one embodiment, the first skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. Alternatively, the first skirt may be formed from a suitable natural or biological graft material such as pericardium or another membranous tissue including, but not limited to intestinal submucosa.

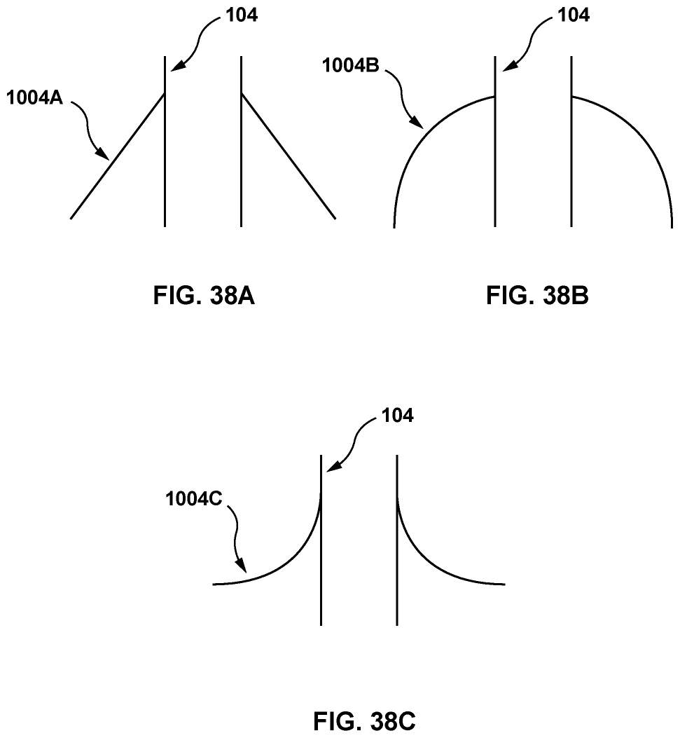

FIGS. 27-37 illustrate various embodiments of different anchoring mechanisms that may be utilized to improve sealing and migration resistance of an anchoring frame. More particularly, FIGS. 27-28 illustrate the tissue engaging elements 514A as an anchoring mechanism, FIGS. 29-30 illustrate a combination of the tissue engaging elements 614A and the distal extension 685 as an anchoring mechanism, FIGS. 31-32 illustrate a combination of the tissue engaging elements 714A and the distal extension 785 as an anchoring mechanism, FIGS. 33-35 illustrate the distal extension 885 as an anchoring mechanism, and FIGS. 36-37 illustrate clip(s) 990 as an anchoring mechanism. For illustrative purposes only, such anchoring mechanisms are shown on an anchoring frame that is the same as or similar to the anchoring frame 302 of FIGS. 15-21. However, it will be apparent to those of ordinary skill in the art that the anchoring mechanisms of FIGS. 27-37 may be incorporated onto anchoring frames having other configurations or shape profiles, such as but not limited the anchoring frame 102 and the anchoring frame 402 described herein. Further, FIGS. 38A, 38B, 38C illustrate anchoring frames 1004A, 1004B, 1004C, respectively, having other exemplary shape profiles that may or may not incorporate the anchoring mechanisms of FIGS. 27-37 thereon to improve sealing and migration resistance of an anchoring frame. These and similar anchoring-frame profiles may be utilized in other embodiments of the prostheses shown in any of the previous figures, or variations thereof. For example, FIG. 38A shows an anchoring frame 1004A with a steepled profile, which forms an acute angle at its attachment point to valve support 104. The attachment point is depicted between the ends of valve support 104 in FIG. 38A, but may, in some embodiments, be placed at any location along the longitudinal edge of valve support 104. It may be conical, D-shaped, or otherwise shaped to conform to the native anatomy at the desired implant location.

As another example, FIG. 38B. shows an anchoring frame 1004B with a dome-like or hemispherical profile, which forms an arc depicted as downward from its attachment point to valve support 104 toward the end of valve support 104 depicted as the bottom of FIG. 38. The attachment point is depicted between the ends of valve support 104 in FIG. 38B, but may, in some embodiments, be placed at any location along the longitudinal edge of valve support 104. It may be hourglass shaped, S-shaped, D-shaped, or otherwise shaped to conform to the native anatomy at the desired implant location.

To further illustrate the various shapes contemplated with respect to FIG. 38B, above, FIG. 38C shows one example, an anchoring frame 1004A with an outwardly flaring profile. Its profile forms an arc starting from its attachment point to valve support 104 that slopes gradually laterally, away from valve support 104 as it extends toward the end of valve support 104 depicted at the bottom of FIG. 38C. The attachment point is depicted between the ends of valve support 104, but may, in some embodiments, be placed at any location along the longitudinal edge of valve support 104. It may be hourglass shaped, S-shaped, D-shaped, or otherwise shaped to conform to the native anatomy at the desired implant location.

In accordance with a first example hereof, a transcatheter heart valve prosthesis includes an anchoring frame configured for placement within a native atrioventricular valve. The anchoring frame includes an outflow end and an inflow end. The anchoring frame has a bell-shaped profile which includes a fixation portion and a lateral portion. The lateral portion flares radially outward, in a direction from the inflow end to the outflow end, at a first angle relative to a central longitudinal axis of the anchoring frame, the first angle being between 40° and 60°. The fixation portion flares radially outward, in a direction from the inflow end to the outflow end, at a second angle relative to the central longitudinal axis of the anchoring frame, the second angle being between 10° and 30°.

In a second example, in the transcatheter heart valve prosthesis according to any of the previous or subsequent examples herein, the fixation portion is a first fixation portion and the anchoring frame further includes a second fixation portion that is distal to the first fixation portion. The second fixation portion flares radially outward, in a direction from the inflow end to the outflow end, at a third angle relative to the central longitudinal axis of the anchoring frame, the third angle being between 30° and 50°.

In a third example, in the transcatheter heart valve prosthesis according to any of the previous or subsequent examples herein, a first plurality of tissue engaging elements is disposed on an outer wall of the anchoring frame and extending towards the inflow end and radially outward, the first plurality of tissue engaging elements disposed on a node or an endmost outflow crown of the anchoring frame along the fixation portion.

In a fourth example, in the transcatheter heart valve prosthesis according to any of the previous or subsequent examples herein, a second plurality of tissue engaging elements is disposed on an outer wall of the anchoring frame and extending towards the inflow end and radially outward, the second plurality of tissue engaging elements disposed on a node or an endmost outflow crown at the outflow end of the anchoring frame. Each tissue engaging element of the second plurality of tissue engaging elements has a hooked shape.

In a fifth example, in the transcatheter heart valve prosthesis according to any of the previous or subsequent examples herein, the second plurality of tissue engaging elements extends around a circumference of the anchoring frame, at the outflow end of the anchoring frame.

In a sixth example, in the transcatheter heart valve prosthesis according to any of the previous or subsequent examples herein, the second plurality of tissue engaging elements does not extend around a circumference of the anchoring frame.

In a seventh example, in the transcatheter heart valve prosthesis according to any of the previous or subsequent examples herein, the second plurality of tissue engaging elements extends around between 135° and 170° of the circumference of the anchoring frame.

In an eighth example, in the transcatheter heart valve prosthesis according to any of the previous or subsequent examples herein, the anchoring frame includes a distal extension of the fixation portion at the outflow end that includes an additional row of cells that does not extend around a circumference of the outflow end of the anchoring frame.

In a ninth example, in the transcatheter heart valve prosthesis according to any of the previous or subsequent examples herein, the distal extension extends around between 190° and 225° of the circumference of the outflow end of the anchoring frame.

In a tenth example, in the transcatheter heart valve prosthesis according to any of the previous or subsequent examples herein, the distal extension extends around between 30° and 60° of the circumference of the outflow end of the anchoring frame.

In an eleventh example, in the transcatheter heart valve prosthesis according to any of the previous or subsequent examples herein, the distal extension has a radially compressed state in which it is generally straightened and distally extends from the fixation portion of the anchoring frame and has a radially expanded state in which it extends generally perpendicular to the central longitudinal axis of the anchoring frame.

In a twelve example, in the transcatheter heart valve prosthesis according to any of the previous or subsequent examples herein, at least one clip is disposed at the outflow end of the anchoring frame, wherein in a radially expanded state of the clip, the clip includes a pair of hinged curved portions. Each curved portion extends radially outward relative to the anchoring frame, and is integrally formed with a U-shaped body of the clip that proximally extends from the curved portions in an upstream direction. The U-shaped body includes a peak and a pair of legs extending between the peak and curved portions. The peak forms a proximal-most end of the clip.

In a thirteenth example, in the transcatheter heart valve prosthesis according to any of the previous or subsequent examples herein, when the clip is in the radially deployed state, the peak is disposed at a similar longitudinal depth as a junction between the fixation portion and the lateral portion of the anchoring frame.

In a fourteenth example, in the transcatheter heart valve prosthesis according to any of the previous or subsequent examples herein, the at least one clip is a single clip.

In a fifteenth example, in the transcatheter heart valve prosthesis according to any of the previous or subsequent examples herein, the at least one clip is a pair of diametrically opposing clips.

In accordance with a sixteenth example hereof, a transcatheter heart valve prosthesis includes a valve support, a prosthetic valve mounted within the valve support, and an anchoring frame at least partially surrounding the valve support. The anchoring frame and the valve support are attached to each other at respective inflow ends thereof. The anchoring frame includes a fixation portion configured to securely fix the anchoring frame to tissue at a native heart valve, an integration region configured to integrate the anchoring frame with the valve support, and a lateral portion extending between the fixation portion and the integration region. A brim is coupled to and extends radially outwardly from the anchoring frame at a transition between the lateral portion and the fixation portion.

In a seventeenth example, in the transcatheter heart valve prosthesis according to any of the previous or subsequent examples herein, the brim includes two sinusoidal rings and a sealing component disposed over or covering at least a downstream surface of the sinusoidal rings.

In an eighteenth example, in the transcatheter heart valve prosthesis according to any of the previous or subsequent examples herein, the transcatheter heart valve prosthesis also includes a first skirt coupled to an inner surface of the anchoring frame and a second skirt coupled to an inner surface of the valve support. The first skirt runs along the inner surface of the anchoring frame, folds where the anchoring frame meets the valve support, and a proximal portion thereof is attached to an outer surface of the valve support.

In a nineteenth example, in the transcatheter heart valve prosthesis according to any of the previous or subsequent examples herein, the prosthetic valve includes two or more leaflets and a scalloped outflow end of the second skirt is attached to inflow ends of the leaflets to form a joint line.

In a twentieth example, in the transcatheter heart valve prosthesis according to any of the previous or subsequent examples herein, the first skirt includes a ring skirt which is cylindrical and a cone skirt.

In a twenty-first example, in the transcatheter heart valve prosthesis according to any of the previous or subsequent examples herein, the first skirt is a single piece skirt.

In a twenty-second example, in the transcatheter heart valve prosthesis according to any of the previous or subsequent examples herein, the lateral portion flares radially outward, in a direction from the inflow end to the outflow end, at a first angle relative to a central longitudinal axis of the anchoring frame, the first angle being between 40° and 60°.

In a twenty-third example, in the transcatheter heart valve prosthesis according to any of the previous or subsequent examples herein, the anchoring frame has a bell-shaped profile and the fixation portion flares radially outward, in a direction from the inflow end to the outflow end, at a second angle relative to the central longitudinal axis of the anchoring frame, the second angle being between 10° and 30°.

In a twenty-fourth example, in the transcatheter heart valve prosthesis according to any of the previous or subsequent examples herein, the anchoring frame has a first outer diameter at the inflow end, a second diameter at a junction between the lateral portion and the fixation portion, and a third outer diameter at an outflow end of the anchoring frame, the second outer diameter being greater than the first outer diameter and the third outer diameter being greater than the second outer diameter.

In a twenty-fifth example, in the transcatheter heart valve prosthesis according to any of the previous or subsequent examples herein, the fixation portion is a first fixation portion and the anchoring frame further includes a second fixation portion that is distal to the first fixation portion. The second fixation portion flares radially outward, in a direction from the inflow end to the outflow end, at a third angle relative to the central longitudinal axis of the anchoring frame, the third angle being between 30° and 50°.

In a twenty-sixth example, in the transcatheter heart valve prosthesis according to any of the previous or subsequent examples herein, the anchoring frame has a first outer diameter at the inflow end, a second diameter at a junction between the lateral portion and the first fixation portion, a third outer diameter at a junction between the first fixation portion and the second fixation portion, and a fourth outer diameter at an outflow end of the anchoring frame, the second outer diameter being greater than the first outer diameter the third outer diameter being greater than the second outer diameter, and the fourth outer diameter being greater than the third outer diameter.

In a twenty-seventh example, in the transcatheter heart valve prosthesis according to any of the previous or subsequent examples herein, the transcatheter heart valve prosthesis also includes a first plurality of tissue engaging elements is disposed on an outer wall of the anchoring frame and extending towards the inflow end and radially outward, the first plurality of tissue engaging elements disposed on a node or an endmost outflow crown of the anchoring frame along the fixation portion.

In a twenty-eighth example, in the transcatheter heart valve prosthesis according to any of the previous or subsequent examples herein, a second plurality of tissue engaging elements is disposed on an outer wall of the anchoring frame and extending towards the inflow end and radially outward, the second plurality of tissue engaging elements disposed on a node or an endmost outflow crown at the outflow end of the anchoring frame. Each tissue engaging element of the second plurality of tissue engaging elements has a hooked shape.

In a twenty-nineth example, in the transcatheter heart valve prosthesis according to any of the previous or subsequent examples herein, the second plurality of tissue engaging elements extends around a circumference of the anchoring frame, at the outflow end of the anchoring frame.

In a thirtieth example, in the transcatheter heart valve prosthesis according to any of the previous or subsequent examples herein, the second plurality of tissue engaging elements extends around between 135° and 170° of a circumference of the anchoring frame.

In a thirty-first example, in the transcatheter heart valve prosthesis according to any of the previous or subsequent examples herein, the anchoring frame includes a distal extension of the fixation portion at the outflow end that includes an additional row of cells that does not extend around a circumference of the outflow end of the anchoring frame. The distal extension does not circumferentially overlap with the second plurality of tissue engaging elements, and the distal extension and the second plurality of tissue engaging elements collectively extend the circumference of the anchoring frame.

In a thirty-second example, in the transcatheter heart valve prosthesis according to any of the previous or subsequent examples herein, a V-shaped connector circumferentially extends between pairs of adjacent tissue engaging elements of the second plurality of tissue engaging elements, the V-shaped connector including an apex and a pair of legs that distally extend from the apex.

In a thirty-third example, in the transcatheter heart valve prosthesis according to any of the previous or subsequent examples herein, the anchoring frame includes a distal extension of the fixation portion at the outflow end that includes an additional row of cells that does not extend around a circumference of the outflow end of the anchoring frame. The distal extension has a radially compressed state in which it is generally straightened and distally extends from the fixation portion of the anchoring frame and has a radially expanded state in which it extends generally perpendicular to the central longitudinal axis of the anchoring frame.

In a thirty-fourth example, in the transcatheter heart valve prosthesis according to any of the previous or subsequent examples herein, the transcatheter heart valve prosthesis further includes at least one clip disposed at the outflow end of the anchoring frame. In a radially expanded state of the clip, the clip includes a pair of hinged curved portions. Each curved portion extends radially outward relative to the anchoring frame and is integrally formed with a U-shaped body of the clip that extends from the curved portions in an upstream direction. The U-shaped body includes a peak and a pair of legs extending between the peak and curved portions. The peak forms a proximal-most end of the clip. When the clip is in the radially deployed state, the peak is disposed at a similar longitudinal depth as a junction between the fixation portion and the lateral portion of the anchoring frame.

In accordance with a thirty-fifth example hereof, a transcatheter heart valve prosthesis includes a valve support, a prosthetic valve mounted within the valve support, and an anchoring frame at least partially surrounding the valve support. The anchoring frame and the valve support are attached to each other at respective outflow ends thereof. The anchoring frame includes a fixation portion configured to securely fix the anchoring frame to tissue at a native heart valve, an integration region configured to integrate the anchoring frame with the valve support, and a lateral portion extending between the fixation portion and the integration region. A brim is coupled to and extends radially outwardly from the fixation portion of the anchoring frame at a brim attachment location. A sealing zone of the fixation portion extends between an inflow end of the anchoring frame and the brim attachment location. At least two rows of barbs are disposed on an outer surface of the anchoring frame along the fixation portion, each barb extending radially outward and towards the inflow end of the anchoring frame. A row of safety cleats is disposed on the outer surface of the anchoring frame along the lateral portion, each barb extending radially outward and generally perpendicular to a central longitudinal axis of the anchoring frame.

Various aspects disclosed herein may be combined in different combinations than the combinations specifically presented in the description and accompanying drawings. Depending on the example or embodiment, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. A transcatheter heart valve prosthesis comprising:
a valve support;
a prosthetic valve mounted within the valve support;
an anchoring frame at least partially surrounding the valve support, wherein the anchoring frame and the valve support are attached to each other at respective inflow ends thereof, wherein the anchoring frame includes a fixation portion configured to securely fix the anchoring frame to tissue at a native heart valve, an integration region configured to integrate the anchoring frame with the valve support, and a lateral portion extending between the fixation portion and the integration region; and
a brim coupled to and extending radially outwardly from the anchoring frame at a transition between the lateral portion and the fixation portion.

2. The transcatheter valve prosthesis of claim 1, wherein the brim includes two sinusoidal rings and a sealing component disposed over or covering at least a downstream surface of the sinusoidal rings.

3. The transcatheter valve prosthesis of claim 1, further comprising a first skirt coupled to an inner surface of the anchoring frame and a second skirt coupled to an inner surface of the valve support, wherein the first skirt runs along the inner surface of the anchoring frame, folds where the anchoring frame meets the valve support, and a proximal portion thereof is attached to an outer surface of the valve support.

4. The transcatheter valve prosthesis of claim 3, wherein the prosthetic valve includes two or more leaflets and wherein a scalloped outflow end of the second skirt is attached to inflow ends of the leaflets to form a joint line.

5. The transcatheter valve prosthesis of claim 3, wherein the first skirt includes a ring skirt which is cylindrical and a cone skirt.

6. The transcatheter valve prosthesis of claim 3, wherein the first skirt is a single piece skirt.

7. The transcatheter valve prosthesis of claim 1, wherein the lateral portion flares radially outward, in a direction from the inflow end to the outflow end, at a first angle relative to a central longitudinal axis of the anchoring frame, the first angle being between 40° and 60°.

8. The transcatheter valve prosthesis of claim 1, wherein the anchoring frame has a bell-shaped profile and the fixation portion flares radially outward, in a direction from the inflow end to the outflow end, at a second angle relative to a central longitudinal axis of the anchoring frame, the second angle being between 10° and 30°.

9. The transcatheter valve prosthesis of claim 8, wherein the anchoring frame has a first outer diameter at the inflow end, a second diameter at a junction between the lateral portion and the fixation portion, and a third outer diameter at an outflow end of the anchoring frame, the second outer diameter being greater than the first outer diameter and the third outer diameter being greater than the second outer diameter.

10. The transcatheter valve prosthesis of claim 8, wherein the fixation portion is a first fixation portion and the anchoring frame further includes a second fixation portion that is distal to the first fixation portion, and wherein the second fixation portion flares radially outward, in a direction from the inflow end to the outflow end, at a third angle relative to a central longitudinal axis of the anchoring frame, the third angle being between 30° and 50°.

11. The transcatheter valve prosthesis of claim 10, wherein the anchoring frame has a first outer diameter at the inflow end, a second diameter at a junction between the lateral portion and the first fixation portion, a third outer diameter at a junction between the first fixation portion and the second fixation portion, and a fourth outer diameter at an outflow end of the anchoring frame, the second outer diameter being greater than the first outer diameter the third outer diameter being greater than the second outer diameter, and the fourth outer diameter being greater than the third outer diameter.

12. The transcatheter valve prosthesis of claim 1, further comprising a first plurality of tissue engaging elements is disposed on an outer wall of the anchoring frame and extending towards the inflow end and radially outward, the first plurality of tissue engaging elements disposed on a node or an endmost outflow crown of the anchoring frame along the fixation portion.

13. The transcatheter valve prosthesis of claim 12, further comprising a second plurality of tissue engaging elements is disposed on an outer wall of the anchoring frame and extending towards the inflow end and radially outward, the second plurality of tissue engaging elements disposed on a node or an endmost outflow crown at the outflow end of the anchoring frame, wherein each tissue engaging element of the second plurality of tissue engaging elements has a hooked shape.

14. The transcatheter valve prosthesis of claim 13, wherein the second plurality of tissue engaging elements extends around a circumference of the anchoring frame, at the outflow end of the anchoring frame.

15. The transcatheter valve prosthesis of claim 13, wherein the second plurality of tissue engaging elements extends around between 135° and 170° of a circumference of the anchoring frame.

16. The transcatheter valve prosthesis of claim 15, wherein the anchoring frame includes a distal extension of the fixation portion at the outflow end that includes an additional row of cells that does not extend around a circumference of the outflow end of the anchoring frame, and wherein the distal extension does not circumferentially overlap with the second plurality of tissue engaging elements, and wherein the distal extension and the second plurality of tissue engaging elements collectively extend the circumference of the anchoring frame.

17. The transcatheter valve prosthesis of claim 16, wherein a V-shaped connector circumferentially extends between pairs of adjacent tissue engaging elements of the second plurality of tissue engaging elements, the V-shaped connector including an apex and a pair of legs that distally extend from the apex.

18. The transcatheter valve prosthesis of claim 1, wherein the anchoring frame includes a distal extension of the fixation portion at the outflow end that includes an additional row of cells that does not extend around a circumference of the outflow end of the anchoring frame, and wherein the distal extension has a radially compressed state in which it is generally straightened and distally extends from the fixation portion of the anchoring frame and has a radially expanded state in which it extends generally perpendicular to the central longitudinal axis of the anchoring frame.

19. The transcatheter valve prosthesis of claim 1, further comprising at least one clip disposed at the outflow end of the anchoring frame, wherein in a radially expanded state of the clip, the clip includes a pair of hinged curved portions, each curved portion extending radially outward relative to the anchoring frame and being integrally formed with a U-shaped body of the clip that extends from the curved portions in an upstream direction, the U-shaped body including a peak and a pair of legs extending between the peak and curved portions, wherein the peak forms a proximal-most end of the clip and wherein when the clip is in the radially deployed state, the peak is disposed at a similar longitudinal depth as a junction between the fixation portion and the lateral portion of the anchoring frame.

20. A transcatheter heart valve prosthesis comprising:
a valve support;
a prosthetic valve mounted within the valve support;
an anchoring frame at least partially surrounding the valve support, wherein the anchoring frame and the valve support are attached to each other at respective outflow ends thereof, wherein the anchoring frame includes a fixation portion configured to securely fix the anchoring frame to tissue at a native heart valve, an integration region configured to integrate the anchoring frame with the valve support, and a lateral portion extending between the fixation portion and the integration region;
a brim coupled to and extending radially outwardly from the fixation portion of the anchoring frame at a brim attachment location, wherein a sealing zone of the fixation portion extends between an inflow end of the anchoring frame and the brim attachment location;
at least two rows of barbs disposed on an outer surface of the anchoring frame along the fixation portion, each barb extending radially outward and towards the inflow end of the anchoring frame; and
a row of safety cleats disposed on the outer surface of the anchoring frame along the lateral portion, each barb extending radially outward and generally perpendicular to a central longitudinal axis of the anchoring frame.
